(19) United States
(12) Patent Application Publication
(10) Pub. No.: US 2024/0150307 A1
(43) Pub. Date: May 9, 2024

(54) MODERATOR AND CATALYST PERFORMANCE OPTIMIZATION FOR EPOXIDATION OF ETHYLENE

(71) Applicant: SHELL USA, INC., Houston, TX (US)

(72) Inventors: Gary James WELLS, Houston, TX (US); Randall Clayton YEATES, Houston, TX (US)

(21) Appl. No.: 18/549,918

(22) PCT Filed: Apr. 6, 2022
(86) PCT No.: PCT/EP2022/059114

§ 371 Date: Sep. 10, 2023

(30) Foreign Application Priority Data
Apr. 8, 2021 (EP) 21167333.0

(51) Int. Cl. C07D 301/10; G16C 20/10

(52) U.S. Cl. CPC C07D 301/10; G16C 20/10

(57) ABSTRACT

A method for maximizing the selectivity (S) of an epoxidation catalyst in an ethylene oxide reactor system, comprising: receiving a measured reactor selectivity (Sₘₑₐₛ), a measured reactor temperature (Tₘₑₐₛ), and one or more operational parameters from an ethylene oxide production system, the measured reactor selectivity (Sₘₑₐₛ), the measured reactor temperature (Tₘₑₐₛ), and the one or more operational parameters comprise real-time and historical operating data points over time generated by the ethylene oxide production system, and using a processor to conduct various calculations and determination in order to output an actionable recommendation that includes a target change (M_{change}) of a moderator level (M) of a chloride-containing catalyst moderator to its optimal value (Mₒₚₜ). The method further includes using the processor to (f) display the actionable recommendation on a display.

The present invention generally relates to determining maximum catalyst selectivity for the epoxidation of ethylene. More specifically, the present invention relates to a system and method that determines an optimum moderator level that achieves maximum catalyst selectivity in real-time.

### BACKGROUND OF THE INVENTION

Ethylene oxide (EO) is a valuable chemical product that is well-known for its use as a versatile chemical intermediate in the production of a wide variety of chemicals and products. For example, EO is often used to produce ethylene glycol, which is used in many diverse applications and may be found in a variety of products, including automotive engine antifreeze, hydraulic brake fluids, resins, fibers, solvents, paints, plastics, films, household and industrial cleaners, pharmaceutical preparations and personal care items such as cosmetics and shampoos, among others.

In the commercial production of EO, ethylene (C₂H₄) is reacted with oxygen (O₂) in the presence of a silver-based ethylene epoxidation catalyst. The catalyst performance may be assessed on the basis of selectivity, activity and stability of operation. The selectivity (S) of the ethylene epoxidation catalyst, also known as the “efficiency,” refers to the ability of the ethylene epoxidation catalyst to convert ethylene to the desired reaction product (i.e., EO) versus the competing by-products (i.e., carbon dioxide (CO₂) and water (H₂O)). The activity refers to the rate of the epoxidation reaction and is usually described as the temperature (T) required to maintain a given production rate of EO by the epoxidation catalyst. The stability of the ethylene epoxidation catalyst refers to how the selectivity and/or activity of the process changes during the time a charge of catalyst is being used, that is, as more EO is produced over time.

There are various approaches to improve the performance of ethylene epoxidation catalysts, which include improvements in selectivity, activity and stability. For example, certain silver-based ethylene epoxidation catalysts, often referred to as “high-selectivity” catalysts, include a rhenium (Re) promoter in addition to silver, as disclosed for example in U.S. Pat. Nos. 4,761,394 A and 4,766,105 A. Optionally, certain silver-based ethylene epoxidation catalysts may also include one or more additional promoters such as alkali metals (e.g., cesium and lithium), alkaline earth metals (e.g., magnesium), transition metals (e.g., tungsten) and main group non-metals (e.g., sulfur). Additionally, beyond improvements in catalyst formulation, catalyst moderators, also commonly referred to as reaction modifiers, have been found that may be added to a reactor feed gas to improve selectivity. Such moderators suppress the undesirable oxidation of ethylene or EO to CO₂ and water, relative to the desired formation of EO. Suitable catalyst moderators for high-selectivity silver epoxidation catalysts are, for example, organic halides, such as methyl chloride, ethyl chloride, ethylene dichloride or vinyl chloride.

However, while the addition of a catalyst moderator generally improves the performance of high-selectivity silver epoxidation catalyst, i.e. a catalyst having silver (Ag), rhenium (Re) and one or more alkali metal promoters on a solid refractory support, these catalysts nevertheless age over time and their activity decreases. Accordingly, as the catalyst ages, the epoxidation reaction temperature is increased over time to maintain ethylene oxide production at the desired level.

Moreover, when using many high selectivity silver epoxidation catalysts, the moderator concentration in the reactor feed gas (e.g., the feed gas entering a EO reactor) must be adjusted to maintain maximum catalyst selectivity as the operating conditions such as the EO production parameter, gas hourly space velocity (GHSV), reactor inlet pressure, and reactor feed concentrations of O₂, C₂H₄, CO₂, and H₂O change, as discussed for example in EP 0352850 A1, U.S. Pat. Nos. 7,193,094 B2, 8,362,284 B2, WO 2010/123842 A1, U.S. Pat. Nos. 9,221,776 B2 and 10,208,005 B2.

When applying a moderator, it is generally accepted that the moderator concentration in the reactor feed gas should be chosen such that the catalyst selectivity is maintained at the maximum value. The underlying chemistry that determines the optimum moderator levels depends on the surface concentration of chlorides rather than the gas phase concentration. The surface concentration of chlorides is the result of adsorption and desorption phenomena, which in turn, depend on many factors. Important factors include the gas-phase concentrations of the moderator species, the catalyst surface concentration of the catalyst dopants, the gas-phase concentrations of hydrocarbons that may remove the chlorides, the reaction temperature, other species concentrations that affect the catalyst surface coverage, and the dynamics of chloride adsorption/desorption that may take hours or longer.

Operators of EO catalyst use various methods to introduce and control the level of chlorides on the catalyst. The moderator level (M) can be controlled by measuring and varying the feed concentration of chlorides or by the fresh feed rate of the moderator to the reactor. Other constructs have been used in control of the moderator level to normalize the chloride levels with respect to hydrocarbons that can remove them from the catalysts.

Another method of defining and controlling the moderator level is to account for the impacts of hydrocarbon concentrations on the surface chloride levels by using an effective chloride level that applies a ratio of a weighted sum of the gas-phase chloride concentrations to a weighted sum of hydrocarbon concentrations, as disclosed in WO 03/044002 A1 and WO 2005/035513 A1. These approaches capture the steady-state impacts of changes in gas-phase chlorides and hydrocarbon concentrations on the equilibrium chloride concentration on the catalyst surface. However, other factors such as temperature and operating condition changes can also affect the surface chloride concentration and the optimum moderator levels.

Existing techniques for optimizing the moderator level (M) include gradually changing (i.e., stepping) the moderator level (M) periodically and observing the selectivity and activity response of the catalyst. The point of maximum selectivity (Sₒₚₜ) is typically chosen after this stepping optimization. The moderator level (M) at which the point of maximum selectivity (Sₒₚₜ) is obtained is referred to as the “optimum” moderator level (Mₒₚₜ). The process is repeated periodically or when significant changes to the operating conditions occur. However, stepping the moderator level (M) is a manual process performed by an operator of an EO production system, which may be tedious and inefficient. Additionally, it may be difficult to gauge if the change in the moderator level (M) is sufficient to observe an improvement in the selectivity of the catalyst that is outside of the overall noise of the process, which is complicated by changes in the operating conditions. Delays between changes in the moderator level (M) and full equilibrium of the surface of the catalyst and the effect on catalyst performance may also present challenges. Further, accurate measurement of the moderator concentration in the feed gas or the normalized gas phase chloride concentration may be difficult, especially in the industrial plant environment, resulting in less reliable optimization of the moderator level.

Certain existing moderator level optimization techniques include monitoring the gas-phase moderator concentration in the reactor feed gas or a ratio of a weighted sum of chloride concentrations to a weighted sum of hydrocarbon concentrations, and relating the optimum level to the temperature. For example, U.S. Pat. No. 7,193,094 B2 discloses a process that depends on changes in temperature and a ratio of an effective molar quantity of active moderator species (i.e., chlorides) in a feed gas to an effective molar quantity of hydrocarbons present in the feed gas. Similarly, U.S. Pat. No. 9,221,776 B2 correlates, via an exponential relationship, a change in a concentration of the moderator with a change in the temperature to maintain maximum catalyst selectivity (Sₒₚₜ). For example, in U.S. Pat. No. 9,221,776 B2, the moderator level is adjusted each time the temperature changes without considering other factors that may affect maximum catalyst selectivity (Sₒₚₜ).

These methods of selecting the optimal moderator levels (Mₒₚₜ) have limitations that reduce their applicability in an industrial EO unit. For example, one limitation is that the methods require accurate and precise measurement of gas phase chlorides, which can be difficult to achieve in an industrial plant environment. Also, the gas-phase chloride concentrations, or the normalized forms such as defined in WO 03/044002 A1, U.S. Pat. No. 7,193,094 B2, or WO 2005/035513 A1, are not always indicative of the surface chloride levels that determine catalyst performance. The dynamics of adsorption and desorption may take several hours to days, meaning that there is a delay in the effects on catalyst performance. The dynamics of adsorption and desorption complicate optimization in the industrial plant environment. Finally, even at steady-state temperature and hydrocarbon concentrations, there are other factors that influence optimal chloride levels. By way of non-limiting example, factors such as non-hydrocarbon species concentrations (e.g., CO₂) and catalyst age may impact the optimum chloride levels on the catalyst surface in addition to their impact on temperature.

U.S. Pat. No. 9,174,928 B2 describes a process for the epoxidation of ethylene comprising:
(a) following start-up, contacting an epoxidation catalyst comprising silver and a rhenium promoter with a feed composition comprising a first concentration of ethylene, a first concentration of oxygen, a first concentration of carbon dioxide that is below 2.0 vol. %, and a first concentration of chloride moderator to achieve a desired work rate W₁ at a first operating temperature;
(b) subsequent to step (a), adjusting the feed composition while maintaining the desired work rate W₁ so as to increase the first operating temperature to a second operating temperature, wherein adjusting the feed composition comprises one or more of the following:
(i) decreasing the first concentration of ethylene to a second concentration of ethylene,
(ii) decreasing the first concentration of oxygen to a second concentration of oxygen,
(iii) increasing the first concentration of carbon dioxide to a second concentration of carbon dioxide, and (iv) decreasing or increasing the first concentration of the chloride moderator to a second concentration of the chloride moderator; and
(c) subsequent to step (b), further adjusting the feed composition so as to maintain the desired work rate W₁ at the second operating temperature, wherein further adjusting the feed composition comprises one or more of the following:
(i) increasing the second concentration of ethylene to a third concentration of ethylene,
(ii) increasing the second concentration of oxygen to a third concentration of oxygen,
(iii) decreasing the second concentration of carbon dioxide to a third concentration of carbon dioxide, and
(iv) increasing or decreasing the second concentration of the chloride moderator to a third concentration of the chloride moderator.

The method described in U.S. Pat. No. 9,174,928 B2 provides guidance on modifying conditions to obtain a temperature that maximizes selectivity for a given work rate by changing conditions such as ethylene concentration or oxygen concentration. The moderator level also needs to be changed to maintained optimum selectivity, but no specific guidance on the appropriate optimal level is given by the method.

U.S. Pat. No. 8,362,284 B2 discloses a process that focuses on determining changes in either temperature or a chloriding effectiveness parameter (Z*) associated with the concentration of the moderator to achieve a desired EO production level or some other desired target. However, this technique does not establish how to determine an initial optimum moderator level in the catalyst run. Rather, this approach first assumes optimal operation and then merely assesses whether the chlorides remain near optimum after a condition change, and it provides guidance on the change required to re-attain an optimum chloride level. Additionally, this technique requires varying only one of two parameters (i.e., the temperature or Z*) at a time while other process conditions (e.g., GHSV, pressure, feed gas composition, etc.) are held substantially fixed. In normal industrial EO production, these other conditions often vary over time due to intentional changes or process disturbances. As such, this technique is not robust to disturbances that occur during normal EO production plant operation.

WO 2016/108975 A1 estimates a direction of chloride optimum vs a pre-determined optimum condition when operating conditions change such as the EO production rate. Similar to the other techniques, WO 2016/108975 A1 requires knowledge of an optimum condition during the catalyst run prior to the change in an operating condition. Additionally, this technique is limited to the use of data collected over a short period of time (e.g., 7 days). Although the information obtained from this technique is directional (e.g., indicating whether the catalyst is overmoderated or undermoderated), it does not provide a magnitude that the moderator level should be adjusted to in order to achieve maximum catalyst selectivity. Therefore, an operator of the EO production system is left to adjust the moderator level ad hoc in the specified direction to find the moderator level that achieves maximum catalyst selectivity, which is not robust.

U.S. Pat. No. 9,892,238 B2 describes a system for monitoring a process determined by a set of process data in a multidimensional process data domain pertaining to process input-output data, the system comprising:
means for acquiring a plurality of historic process data sets;
means for obtaining a transformation from the multidimensional process data domain to a model data domain of lower dimension by performing multivariate data analysis;
means for transforming a current process data set to a model data set to monitor the process using the obtained transformation; and
means to detect a permanent change of a process characteristic of the process which is no longer captured by the multivariate data analysis based on observing Residuals exceeding a predetermined threshold for a predetermined amount of time.

The system and method of U.S. Pat. No. 9,892,238 B2 applies a combination of artificial neural nets and principal component analysis to distinguish normal from abnormal or previously seen operating modes. One example is the detection of overmoderation of a catalyst in a process for the epoxidation of ethylene. However, once an overmoderated state is detected, U.S. Pat. No. 9,892,238 B2 does not provide specific steps for a plant operator to return to optimal conditions. The plant operator still needs to apply manual interventions to determine and return the system to optimal operating conditions.

Due to the impact of the moderator level on the selectivity of high-selectivity epoxidation catalysts, it may be desirable to have a moderator level optimization technique that accurately and robustly determines, in real-time, the optimum moderator level (Mₒₚₜ) that leads to the maximum selectivity (Sₒₚₜ) of the silver-based ethylene epoxidation catalyst at the current set of operating conditions. In this way, the moderator level may be adjusted by a given amount, either automatically or by an operator, to achieve maximum catalyst selectivity (Sₒₚₜ) rather than stepping the moderator level until the optimum moderator level (Mₒₚₜ) is found. As discussed in further detail below, the present disclosure overcomes the limitations of existing techniques and provides a robust and effective technique for moderator level optimization.

### SUMMARY

In an embodiment, there is provided a method for maximizing the selectivity (S) of an epoxidation catalyst in an ethylene oxide reactor system comprising receiving a measured reactor selectivity (Sₘₑₐₛ), a measured reactor temperature (Tₘₑₐₛ), and one or more operational parameters from an ethylene oxide production system configured to convert, in the ethylene oxide reactor system, a feed gas comprising ethylene and oxygen into ethylene oxide in the presence of the epoxidation catalyst and a chloride-containing catalyst moderator. The epoxidation catalyst comprises silver and a promoting amount of rhenium (Re), and the measured reactor selectivity (Sₘₑₐₛ), the measured reactor temperature (Tₘₑₐₛ), and the one or more operational parameters includes real-time and historical operating data points over time generated by the ethylene oxide production system. The method also includes using a processor to (a) calculate, using a model, for each time point, a model-estimated selectivity (Sₑₛₜ) and a model-estimated temperature (Tₑₛₜ) of the epoxidation catalyst at an optimum moderator level (Mₒₚₜ). The model-estimated selectivity (Sₑₛₜ) and the model-estimated temperature (Tₑₛₜ) are determined based on at least one operational parameter of the one or more operational parameters at said time points, the at least one operational parameter does not include a chloride-containing moderator level, and the model is based, at least in part, on empirical historical data associated with the epoxidation catalyst, the ethylene oxide production system, or both. The method also includes using the processor to (b) determine a difference (ΔS) between the measured reactor selectivity (Sₘₑₐₛ) and the model-estimated selectivity (Sₑₛₜ) and a difference (ΔT) between the measured reactor temperature (Tₘₑₐₛ) and the model-estimated temperature (Tₑₛₜ) for each of the time points, (c) fit a curve to the delta selectivity (ΔS) data points as a function of the corresponding delta temperature (ΔT) data points to obtain a fitted curve, (d) determine a real-time relative effective moderator level (RCl_{eff}_{ᵣₑₐₗ₋ₜᵢₘₑ}) based on the fitted curve and real-time values of ΔS (ΔSᵣₑₐₗ₋ₜᵢₘₑ) and ΔT (ΔTᵣₑₐₗ₋ₜᵢₘₑ) and (e) output an actionable recommendation based on the real-time RCl_{eff} (RCl_{eff}_{ᵣₑₐₗ₋ₜᵢₘₑ}). The real-time RCl_{eff} (RCl_{eff}_{ᵣₑₐₗ₋ₜᵢₘₑ}) is time determined by:
(i) determining a slope of the fitted curve at the real-time values of ΔS (ΔSᵣₑₐₗ₋ₜᵢₘₑ) and ΔT (ΔTᵣₑₐₗ₋ₜᵢₘₑ) and comparing the slope to a reference curve for the epoxidation catalyst, or by
(ii) determining from the fitted curve a maximum ΔS (ΔSₒₚₜ) and a corresponding ΔT (ΔTₒₚₜ) at the maximum ΔS, wherein the ΔSₒₚₜ occurs at the optimum RCl_{eff}, calculating a relative selectivity difference (RSD) by subtracting the ΔSₒₚₜ from the ΔS and a relative temperature difference (RTD) by subtracting the ΔTₒₚₜ from the ΔT, and comparing real-time values of the RSD (RSDᵣₑₐₗ₋ₜᵢₘₑ) and the RTD (RTDᵣₑₐₗ₋ₜᵢₘₑ) to reference curves for the epoxidation catalyst,
or by a combination of said methods (i) and (ii), wherein the reference curves are generated from previous laboratory testing, pilot plant testing, or earlier plant operation that relate the selectivity deviations and temperature deviations versus optimum to the relative effective moderator level (RCl_{eff}) or that relate the slope of the plot of said selectivity deviations plotted against said temperature deviations to the relative effective moderator level (RCl_{eff}). The recommendation includes a target change (M_{change}) of a moderator level (M) to its optimal value (Mₒₚₜ) such that the RCl_{eff} is changed from its real-time value (RCl_{eff}_{ᵣₑₐₗ₋ₜᵢₘₑ}) to the optimum level of 0.0 by definition or the equivalent absolute moderator level target (Mₒₚₜ),
wherein
the RCl_{eff} is defined to be the value of the ratio of the moderator level (M) to the optimum moderator level (Mₒₚₜ) minus one:

RCl_{eff}=(M/Mₒₚₜ)−1
and, wherein the moderator level (M) is defined as a total or weighted total concentration of chloride species in the feed gas to the ethylene oxide reactor system, a makeup feed rate of chlorides or a catalyst chloriding effectiveness value (cl_{eff}), which is calculated as:

$${Cl}_{eff} = \frac{( 0.1 * [ MC ] + [ EC ] + 2 * [ EDC ] + [ VC ] )}{( 0.002 * [ {CH}_{4} ] + [ {C}_{2} {H}_{6} ] + 0.01 * [ {C}_{2} {H}_{4} ] )}$$

whereby [MC], [EC], [EDC], and [VC] are the concentrations in ppmv of methyl chloride (MC), ethyl chloride (EC), ethylene dichloride (EDC), and vinyl chloride (VC), respectively, and [CH₄], [C₂H₆] and [C₂H₄] are the concentrations in mole percent of methane, ethane, and ethylene, respectively, in the feed gas, and wherein the recommended change to bring the moderator level (M) from its real-time moderator level (Mᵣₑₐₗ₋ₜᵢₘₑ) to its optimal moderator level (Mₒₚₜ) and to bring the RCl_{eff} to its optimum level of 0.0 is defined as

M_{change}=(1/(RCl_{eff}_{ᵣₑₐₗ₋ₜᵢₘₑ}+1)−1)*100%,

in percentage terms or, as the equivalent incremental change in moderator level, or equivalently wherein the recommended absolute optimum moderator level target (Mₒₚₜ) is defined as:

Mₒₚₜ=Mᵣₑₐₗ₋ₜᵢₘₑ/(RCl_{eff}_{ᵣₑₐₗ₋ₜᵢₘₑ}+1)

The method further includes using the processor to (f) display the actionable recommendation on a display.

In another embodiment, there is provided one or more tangible, non-transitory, machine-readable media configured to maximize a selectivity (S) of an epoxidation catalyst in an ethylene oxide reactor system and comprising instructions to (a) calculate, using a model, for real-time and historical points over time, a model-estimated selectivity (Sₑₛₜ) and a model-estimated temperature (Tₑₛₜ) of the epoxidation catalyst at an optimum moderator level (Mₒₚₜ) based on at least one operational parameter at said time points from an ethylene oxide production system comprising the ethylene oxide reactor system. The model is based, at least in part, on empirical historical data associated with the epoxidation catalyst, the ethylene oxide production system, or both, wherein the at least one operational parameter does not include a chloride-containing moderator level, and wherein the epoxidation catalyst comprises silver and a promoting amount of rhenium (Re). The one or more tangible, non-transitory, machine-readable media also includes instructions to: (b) determine a difference (ΔS) between a measured reactor selectivity (Sₘₑₐₛ) and the model-estimated selectivity (Sₑₛₜ) and a difference (ΔT) between a measured reactor temperature (Tₘₑₐₛ) and the model-estimated temperature (Tₑₛₜ) for each of the time points, wherein the measured reactor selectivity (Sₘₑₐₛ), the measured reactor temperature (Tₘₑₐₛ), and the one or more operational parameters comprise real-time and historical operating data points over time generated by the ethylene oxide production system at said time points,
(c) fit a curve to the delta selectivity (ΔS) data points as a function of the corresponding delta temperature (ΔT) data points to obtain a fitted curve,
(d) determine a real-time relative effective moderator level (RCl_{eff}_{ᵣₑₐₗ₋ₜᵢₘₑ}) based on the fitted curve and real-time values of ΔS (ΔSᵣₑₐₗ₋ₜᵢₘₑ) and ΔT (ΔTᵣₑₐₗ₋ₜᵢₘₑ) and
(e) output an actionable recommendation based on the real-time RCl_{eff} (RCl_{eff}_{ᵣₑₐₗ₋ₜᵢₘₑ}). The real-time RCl_{eff} (RCl_{eff}_{ᵣₑₐₗ₋ₜᵢₘₑ}) is determined by: (i) determining a slope of the fitted curve at the real-time values of ΔS (ΔSᵣₑₐₗ₋ₜᵢₘₑ) and ΔT (ΔTᵣₑₐₗ₋ₜᵢₘₑ) and comparing the slope to a reference curve for the epoxidation catalyst, or by (ii) determining from the fitted curve a maximum ΔS (ΔSₒₚₜ) and a corresponding ΔT (ΔTₒₚₜ) at the maximum ΔS, wherein the ΔSₒₚₜ occurs at the optimum RCl_{eff}, calculating a relative selectivity difference (RSD) by subtracting the ΔSₒₚₜ from the ΔS and a relative temperature difference (RTD) by subtracting the ΔTₒₚₜ from the ΔT, and comparing real-time values of the RSD (RSDᵣₑₐₗ₋ₜᵢₘₑ) and the RTD (RTDᵣₑₐₗ₋ₜᵢₘₑ) to reference curves for the epoxidation catalyst,
or by a combination of said methods (i) and (ii), wherein the reference curves are generated from previous laboratory testing, pilot plant testing, or earlier plant operation that relate the selectivity deviations and temperature deviations versus optimum to the relative effective moderator level (RCl_{eff}) or that relate the slope of the plot of said selectivity deviations plotted against said temperature deviations to the relative effective moderator level (RCl_{eff}). The recommendation includes a target change (M_{change}) of a moderator level (M) to its optimal value (Mₒₚₜ) such that the RCl_{eff} is changed from its real-time value (RCl_{eff}_{ᵣₑₐₗ₋ₜᵢₘₑ}) to the optimum level of 0.0 by definition or the equivalent absolute moderator level target (Mₒₚₜ),
wherein the recommended change to bring the moderator level (M) from its real-time moderator level (Mᵣₑₐₗ₋ₜᵢₘₑ) to its optimal moderator level (Mₒₚₜ) and to bring the RCl_{eff} to its optimum level of 0.0 is defined as

M_{change}=(1/(RCl_{eff}_{ᵣₑₐₗ₋ₜᵢₘₑ}+1)−1)*100%,
in percentage terms, or as the equivalent incremental change in moderator level, or equivalently, the recommendation includes an absolute optimum moderator level target (Mₒₚₜ) that is defined as:

Mₒₚₜ=Mᵣₑₐₗ₋ₜᵢₘₑ/(RCl_{eff}_{ᵣₑₐₗ₋ₜᵢₘₑ}+1).
The one or more tangible, non-transitory, machine-readable media also includes instructions to (f) display the actionable recommendation on a display.

In a further embodiment, there is provided a system that comprises a reactor disposed in an ethylene oxide production system and having ethylene, oxygen, an epoxidation catalyst, and a chloride-containing catalyst moderator. The reactor is configured to convert the ethylene and the oxygen into ethylene oxide, and the epoxidation catalyst comprises silver and a promoting amount of rhenium (Re). The system also a display and a data processing system that configured to receive a measured reactor selectivity (Sₘₑₐₛ), a measured reactor temperature (Tₘₑₐₛ), and one or more operational parameters from the ethylene oxide production system. The measured reactor selectivity (Sₘₑₐₛ), the measured reactor temperature (Tₘₑₐₛ), and the one or more operational parameters include real-time and historical operating data points over time generated by the ethylene oxide production system, and the data processing system comprises a processor and one or more tangible, non-transitory, machine-readable media comprising instructions that when executed by the processor are configured to (a) calculate, using a model, for each time point, a model-estimated selectivity (Sₑₛₜ) and a model-estimated temperature (Tₑₛₜ) of the epoxidation catalyst at an optimum moderator level (Mₒₚₜ). The model-estimated selectivity (Sₑₛₜ) and temperature (Tₑₛₜ) are determined based on at least one operational parameter of the one or more operational parameters at said time points, wherein the at least one operational parameter does not include a chloride-containing moderator level, and the model is based at least in part on empirical historical data associated with the epoxidation catalyst, the ethylene oxide production system, or both. The one or more tangible, non-transitory, machine-readable media also includes instructions that when executed by the processor may: (b) determine the difference (ΔS) between the measured reactor selectivity (Sₘₑₐₛ) and the model-estimated selectivity (Sₑₛₜ) and the difference (ΔT) between the measured reactor temperature (Tₘₑₐₛ) and the model-estimated temperature (Tₑₛₜ) for each of the time points, (c) fit a curve to the delta selectivity (ΔS) data points as a function of the corresponding delta temperature (ΔT) data points to obtain a fitted curve, (d) determine a real-time relative effective moderator level (RCl_{eff}_{ᵣₑₐₗ₋ₜᵢₘₑ}) based on the fitted curve and real-time values of ΔS (ΔSᵣₑₐₗ₋ₜᵢₘₑ) and ΔT (ΔTᵣₑₐₗ₋ₜᵢₘₑ) and (e) output an actionable recommendation based on the real-time (RCl_{eff}_{ᵣₑₐₗ₋ₜᵢₘₑ}). The real-time RCl_{eff} (RCl_{eff}_{ᵣₑₐₗ₋ₜᵢₘₑ}) is determined by (i) determining a slope of the fitted curve at the real-time values of ΔS (ΔSᵣₑₐₗ₋ₜᵢₘₑ) and ΔT (ΔTᵣₑₐₗ₋ₜᵢₘₑ) and comparing the slope to a reference curve for the epoxidation catalyst, or by (ii) determining from the fitted curve a maximum ΔS (ΔSₒₚₜ) and a corresponding ΔT (ΔTₒₚₜ) at the maximum ΔS, wherein the ΔSₒₚₜ occurs at the optimum RCl_{eff}, calculating a relative selectivity difference (RSD) by subtracting the ΔSₒₚₜ from the ΔS and a relative temperature difference (RTD) by subtracting the ΔTₒₚₜ from the ΔT, and comparing real-time values of the RSD (RSDᵣₑₐₗ₋ₜᵢₘₑ) and the RTD (RTDᵣₑₐₗ₋ₜᵢₘₑ) to reference curves for the epoxidation catalyst, or by a combination of said methods (i) and (ii), wherein the reference curves are generated from previous laboratory testing, pilot plant testing, or earlier plant operation that relate the selectivity deviations and temperature deviations versus optimum to the relative effective moderator level (RCl_{eff}) or that relate the slope of the plot of said selectivity deviations plotted against said temperature deviations to the relative effective moderator level (RCl_{eff}). The recommendation includes a target change (M_{change}) of a moderator level (M) from its real time value (Mᵣₑₐₗ₋ₜᵢₘₑ) to its optimal value (Mₒₚₜ) such that the RCl_{eff} is changed from its real-time value (RCl_{eff}_{ᵣₑₐₗ₋ₜᵢₘₑ}) to the optimum level of 0.0 by definition or the equivalent absolute moderator level target (Mₒₚₜ),
wherein the recommended change to bring the moderator level (M) from its real-time moderator level (Mᵣₑₐₗ₋ₜᵢₘₑ) to its optimal moderator level (Mₒₚₜ) and to bring the RCl_{eff} to its optimum level of 0.0 is defined as

M_{change}=(1/(RCl_{eff}_{ᵣₑₐₗ₋ₜᵢₘₑ}+1)−1)*100%,
in percentage terms, or as the equivalent incremental change in moderator level, or equivalently, the recommendation includes an absolute optimum moderator level target (Mₒₚₜ) that is defined as:

Mₒₚₜ=Mᵣₑₐₗ₋ₜᵢₘₑ/(RCl_{eff}_{ᵣₑₐₗ₋ₜᵢₘₑ}+1).

The one or more tangible, non-transitory, machine-readable media further includes instructions that when executed by the processor may (f) display the actionable recommendation on a display.

Additional features and advantages of exemplary implementations of the disclosure will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

Advantages of the present disclosure may become apparent upon reading the following detailed description and upon reference to the drawings in which:

FIG. 1 is a representative plot of catalyst selectivity (S) and catalyst chloriding effectiveness (Cl_{eff}) over time for manual laboratory-scale optimization of a chloride moderator;

FIG. 2 is a representative plot of temperature (T) and catalyst chloriding effectiveness (Cl_{eff}) over time for the manual laboratory-scale optimization of the chloride moderator;

FIG. 3 is a representative plot of the catalyst selectivity (S) and temperature (T) as a function of the catalyst chloriding effectiveness (Cl_{eff}) derived from a combination of steady points from the plots of FIGS. 1 and 2;

FIG. 4 is a representative plot of relative selectivity (RS) and relative temperature (RT) as a function of relative effective moderator level (RCl_{eff}) derived from the plots of FIGS. 1-3;

FIG. 5 is a representative plot of selectivity (S) as a function of the catalyst chloriding effectiveness (Cl_{eff}) for a compilation of various offline laboratory tests for a given epoxidation catalyst at different operating conditions and ages;

FIG. 6 is a representative plot of temperature (T) as a function of the catalyst chloriding effectiveness (Cl_{eff}) for a compilation of various offline laboratory tests for a given epoxidation catalyst at different operating conditions and ages;

FIG. 7 is a representative plot of the RS as a function of the RCl_{eff} derived from the plot of FIG. 5 with a fitted relationship, in accordance with an embodiment of the present invention;

FIG. 8 is a representative plot of the RT as a function of the RCl_{eff} derived from the plot of FIG. 6 with a fitted relationship, in accordance with an embodiment of the present invention;

FIG. 9 is a representative plot of a reference curve relating the RS and the RT to RCl_{eff} derived from the fitted relationships in FIGS. 7 and 8, in accordance with an embodiment of the present invention;

FIG. 10 is a plot of the RS vs. the RT using the fitted reference curves of FIG. 9, in accordance with an embodiment of the present invention;

FIG. 11 is a representative plot of a slope of the plot of RS vs. RT as a function of RCl_{eff} derived from the plots of FIGS. 9 and 10 over an expanded range, in accordance with an embodiment of the present invention;

FIG. 12 is a zoomed-in view of the plot of FIG. 11 around point (0, 0);

FIG. 13 is a schematic diagram of an ethylene oxide (EO) production system for determining maximum catalyst selectivity and providing an alert/recommendation on adjustment of a catalyst moderator level, in accordance with an embodiment of the present invention;

FIG. 14 is a flow chart of a method used by the EO production system of FIG. 13 to determine the maximum catalyst selectivity (Sₒₚₜ) and provide the alert/recommendation on the adjustment of the catalyst moderator level, in accordance with an embodiment of the present invention;

FIG. 15 is a representative plot of catalyst selectivity (S) and temperature (T) as a function of days on stream using real-time operation data of the system of FIG. 13 and a model, in accordance with an embodiment of the present invention;

FIG. 16 is a representative plot of ΔS as a function of ΔT derived from the data in a recent period of interest of FIG. 15, with a curve fit to the data that are present on both sides of the optimum moderator level (Mₒₚₜ), in accordance with an embodiment of the present invention;

FIG. 17 is a representative plot of a relative selectivity difference (RSD) as a function of a relative temperature difference (RTD) derived from the plot of FIG. 16 with the alert of an overmoderated state, in accordance with an embodiment of the present invention;

FIG. 18 is a decision tree used by the EO production system of FIG. 13 to provide actionable guidance on adjustment of the catalyst moderator level in real-time, in accordance with an embodiment of the present invention;

FIG. 19 is a representative plot of ΔS as a function of the corresponding ΔT generated by the EO production system of FIG. 13, whereby data are present on one side of the optimum moderator level (Mₒₚₜ) with an alert of an undermoderated state, in accordance with an embodiment of the present invention;

FIG. 20 is a representative plot of ΔS as a function of ΔT generated by the EO production system of FIG. 13, whereby a real-time data point is outside a prediction boundary, in accordance with an embodiment of the present invention;

FIG. 21 is a representative plot of ΔS as a function of ΔT generated by the EO production system of FIG. 13, whereby a trend in the data is unclear, in accordance with an embodiment of the present invention;

FIG. 22 is a representative plot of ΔS as a function of ΔT generated by the EO production system of FIG. 13, whereby the slope of the curve at the real-time point is outside of normal reference bounds, with an alert of severe overmoderation, in accordance with an embodiment of the present invention; and

FIG. 23 is a representative plot of ΔS as a function of ΔT generated by the EO production system of FIG. 13, whereby the slope of the curve at the real-time point is near zero, indicating near optimum moderator level (Mₒₚₜ), in accordance with an embodiment of the present invention.

### DETAILED DESCRIPTION

One or more specific embodiments of the present invention will be described below. These described embodiments are examples of the presently disclosed techniques. Additionally, in an effort to provide a concise description of these embodiments, not all features of an actual implementation may be described in the specification. It should be appreciated that in the development of any such actual implementation, as in any engineering or design project, numerous implementation-specific decisions will be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this invention.

When introducing elements of various embodiments of the present invention, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present invention are not intended to be interpreted as excluding the existence of additional embodiments that also incorporate the recited features.

### Definitions

As used herein, the term “activity,” “catalyst activity” or the like is the productivity of the catalyst for making EO. Activity is often quantified using the temperature required to make a certain amount of EO product. If the activity of the catalyst is higher, the temperature required for a given EO production level is lower. Conversely, if the activity is lower, the temperature required for a given EO production level is higher. References to the terms “reactor temperature,” “catalyst temperature,” “temperature” and the like are used interchangeably herein as a metric for the catalyst activity.

As used herein, the terms “selectivity,” “reactor selectivity,” “catalyst selectivity” and the like refer to the ability of the catalyst to convert ethylene (C₂H₄) to the desired reaction product, ethylene oxide, versus the competing by-products (i.e., carbon dioxide (CO₂) and water (H₂O)), and is expressed as the percentage of the number of moles of ethylene oxide produced per number of moles of ethylene consumed in the reactor.

As used herein, the term “silver-based ethylene epoxidation catalyst,” “ethylene epoxidation catalyst,” “epoxidation catalyst,” “high-selectivity epoxidation catalyst” or the like refers to a catalyst on an alumina support that includes silver in the range of 1 to 40% by weight and a promoting amount of rhenium used in the epoxidation of ethylene to ethylene oxide. As used herein, the term “promoting amount” of rhenium (Re) refers to an amount of Re that works effectively to provide an improvement in one or more of the catalytic properties of a subsequently formed catalyst when compared to a catalyst not containing Re. Examples of catalytic properties include, but are not limited to, selectivity, activity and stability (i.e., selectivity and activity decline over time). It is understood by one skilled in the art that one or more of the individual catalytic properties may be enhanced by the “promoting amount” while other catalytic properties may or may not be enhanced or may even be diminished. It is further understood that different catalytic properties may be enhanced at different operating conditions. For example, a catalyst having enhanced selectivity at one set of operating conditions may be operated at a different set of conditions wherein the improvement is exhibited in the activity rather than in the selectivity.

As used herein, the term “ethylene oxide (EO) production parameter” is a measure of the extent to which ethylene oxide is produced during a process for the epoxidation of ethylene. The EO production parameter may be selected from the group that includes product gas ethylene oxide concentration, the change in the number of moles of EO produced from the inlet to the outlet of the reactor, the ethylene oxide production rate, the ethylene oxide production rate per mass of silver loaded into the reactor, the ethylene oxide production rate per catalyst mass (also known as mass work rate (WRm)) and the ethylene oxide production rate per catalyst volume (also known as work rate (WR)). In the present invention, the preferred ethylene oxide production parameter is work rate, although others could equally be chosen without departing from the scope of the present invention. As used herein, the term “work rate” is intended to denote the mass of EO produced per catalyst volume per time and is generally measured in units of kilograms (kg)/cubic meter (m³) of catalyst/hour (h), kg/m³ cat/h.

As used herein, “operating conditions,” “conditions” and the like refer to the collection of measured or controlled variables that include, but are not limited to, reactor inlet pressure, feed gas flow rate or Gas Hourly Space Velocity (GHSV), the feed gas concentrations of ethylene (C₂H₄), oxygen (O₂), carbon dioxide (CO₂), ethane (C₂H₆), methane (CH₄) and water (H₂O) and the EO production parameter. The reaction temperature and the moderator level are not included in the term “operating conditions.” As used herein, the term “operational parameters” refers to the above operating conditions plus the reaction temperature and the moderator level.

As used herein, the terms “real-time data,” “real-time data point” and the like refer to the latest available set of operational parameters and measured selectivity. Symbols appended with the subscript “real-time” denote the specific value of a time varying quantity at the real-time point. The time frame of the real-time data may be instantaneous, hourly averages, shift averages or daily averages. As used herein, the term “historical operating data” and the like refers to sets of operational parameters and measured selectivity collected prior to the real-time data.

As used herein, the terms “moderator level,” “chloride-containing moderator level” and the like are intended to denote a process variable that is varied to change how much organic chlorides are being delivered to a reactor system and catalyst. The moderator level may be any metric that is directly or indirectly indicative of a steady-state level of chloride moderation of the catalyst such as a total or weighted total concentration of chloride species (i.e., moderator concentration) in the feed gas, the makeup feed rate (i.e., volumetric or mass rate) of the chlorides or the catalyst chloriding effectiveness, which takes into account the impact of hydrocarbons on the chlorides moderating ability. By way of non-limiting example, three specific ways of defining the moderator level are:
1) The total weighted moderator concentration in the reactor feed gas:

TotCl=0.1*[MC]+[EC]+2*[EDC]+[VC] (EQ. 1)
2) The catalyst chloriding effectiveness value (Cl_{eff}):

$${Cl}_{eff} = \frac{( 0.1 * [ MC ] + [ EC ] + 2 * [ EDC ] + [ VC ] )}{( 0.002 * [ {CH}_{4} ] + [ {C}_{2} {H}_{6} ] + 0.01 * [ {C}_{2} {H}_{4} ] )} \qquad ( EQ . 2 )$$

whereby [MC], [EC], [EDC], and [VC] are the concentrations in ppmv of methyl chloride (MC), ethyl chloride (EC), ethylene dichloride (EDC), and vinyl chloride (VC), respectively, and [CH₄], [C₂H₆] and [C₂H₄] are the concentrations in mole percent of methane, ethane, and ethylene, respectively, in the feed gas.
3) The makeup feed rate of moderator.
Any of these moderator level metrics, or others which represent the level of chloride moderation of the catalyst, may be manipulated to change the overall chlorides in the reaction system.

As used herein, the term “relative effective moderator level” (RCl_{eff}), “relative moderator level” or the like refers to the moderator level (M) relative to an optimum moderator level (Mₒₚₜ), which is represented by the following formula:

RCl_{eff}=(M/Mₒₚₜ)−1 (EQ. 3)
whereby “M” refers to the moderator level and the subscript “opt” represents a chloride-optimized moderator level.
EQ. 3 may be rearranged to calculate the chloride-optimized moderator level from the moderator level and RCl_{eff}:

Mₒₚₜ=M/(RCl_{eff}+1) (EQ. 4)

The change (M_{change}) required to move the moderator level to the chloride-optimized moderator level, in percentage terms, is given by the following formula, which involves a rearrangement of EQ. 4:

M_{change}=(Mₒₚₜ/M−1)*100%=(1/(RCl_{eff}+1)−1)*100% (EQ. 5)

Said change may also be given as the equivalent incremental change in moderator level that corresponds to the specified percentage change (M_{change}).

As used herein, the terms “chloride-optimized moderator,” “optimized moderator,” “optimal concentration of catalyst moderator,” “optimum” and the like are used interchangeably and refer to the moderator level (Mₒₚₜ) that results in the maximum catalyst selectivity (Sₒₚₜ) at the current operating conditions. As used herein, the term “undermoderated” is intended to denote a catalyst state where chloride levels in a feed gas are below an optimum chloride (i.e., moderator) level. As used herein, the term “overmoderated” is intended to denote a catalyst state where the chloride levels in the feed gas are above the optimum chloride (i.e., moderator) level.

“Relative selectivity” (RS) is the difference between a measured selectivity (Sₘₑₐₛ) and the selectivity (Sₒₚₜ) at the optimal moderator level (Mₒₚₜ), and it is represented by the following formula:

RS=Sₘₑₐₛ−Sₒₚₜ (EQ. 6)

“Relative temperature” (RT) is the difference between a measured temperature (Tₘₑₐₛ) and a temperature (Tₒₚₜ) at the optimal moderator level (Mₒₚₜ) represented by the following formula:

RT=Tₘₑₐₛ−Tₒₚₜ (EQ. 7)

“Delta selectivity” (ΔS) is the difference between the measured selectivity (Sₘₑₐₛ) and a model predicted value for selectivity (Sₑₛₜ) and is represented by the following formula:

ΔS=Sₘₑₐₛ−Sₑₛₜ (EQ. 8)

“Delta temperature” (ΔT) is the difference between the measured temperature (Tₘₑₐₛ) and a model predicted value for temperature (Tₑₛₜ) and is represented by the following formula:

ΔT=Tₘₑₐₛ−Tₑₛₜ (EQ. 9)

The model may be any suitable model that predicts the effects of changing feed gas composition (e.g., O₂, C₂H₄, CO₂, C₂H₆, CH₄, H₂O), GHSV, pressure and EO production parameter on the chloride-optimized selectivity and temperature.

“Relative selectivity difference” (RSD) is represented by the following formula:

RSD=ΔS−ΔSₒₚₜ (EQ. 10)
where ΔS opt is the maximum ΔS value along a fitted curve of ΔS vs. ΔT (see, e.g., FIG. 16), and where RSD is defined to be zero at the maximum selectivity (Sₒₚₜ)

“Relative temperature difference” (RTD) is represented by the following formula:

RTD=ΔT−ΔTₒₚₜ (EQ. 11)
where ΔTₒₚₜ is the value of ΔT corresponding to the maximum ΔS (i.e. ΔS opt) for the fitted curve for ΔS vs. ΔT (see, e.g., FIG. 16), and where RTD is defined to be zero at the optimum point.

### Advantages of the System and Method

The most common and basic method of optimizing the moderator level of a high-selectivity epoxidation catalyst (e.g., an epoxidation catalyst having silver and a promoting amount of Re) is for an operator of an EO production system at an EO plant to manually step the moderator level. As understood by those of ordinary skill in the art, there are many disturbances and changes in operational goals in normal plant operations based on demands that result in system conditions that fluctuate. Therefore, monitoring selectivity and temperature as a function of the moderator (i.e., chlorides) at a plant would result in undesirable noise due, in part, to condition fluctuations that are difficult to separate from the true effects of moderator level changes. To determine maximum catalyst selectivity accurately and reliably, the impact of various operational parameters of the plant (e.g., temperature, EO production parameter, gas hourly space velocity, pressure, feed composition, moderator level, etc.) should be considered. The techniques disclosed herein are not restricted to varying only one operating condition while all other operating conditions remain substantially constant. By using the process and methods disclosed herein, the fluctuations in operating conditions may be accounted for, thereby allowing for the impact of the changes in the moderator level to be clearly observed more reliably and over a longer period of time.

As discussed above, certain existing techniques for optimizing catalyst selectivity require accurate measurements of gas phase chlorides in the industrial EO plant to utilize linear or power-law equation optimum curves of the moderator concentration or effective ratios of weighted sums of the moderator concentrations relative to weighted sums of the hydrocarbon concentrations as a function of temperature. Other techniques determine changes in only one parameter at a time, such as temperature or moderator concentration, to achieve a desired EO production parameter or another target parameter while other parameters remain constant. However, unlike existing techniques, in the present invention it has been surprisingly found that, to optimize catalyst selectivity, it is not necessary to measure gas phase chlorides accurately in the EO plant environment. Rather, it is sufficient to make effective changes in the chloride feed rate without relying on analysis of the gas phase chlorides. Specifically, the techniques disclosed herein use the catalyst performance itself and account for delays between changing the moderator level and its impact on the catalyst performance. This avoids the potential issues of delays in chloride equilibrium on the catalyst surface that could otherwise confuse optimization.

Moreover, using historical operating data of EO plant operation collected beyond, for example 7 days, and reference data collected during offline catalyst testing (e.g., laboratory or pilot plant testing) or from historical EO plant operation, in combination with accounting for variances in operating conditions (e.g., gas hourly space velocity (GHSV), EO production parameter, feed gas composition, pressure) of the EO production plant, the methods disclosed herein provide a robust, efficient and accurate method for maximizing catalyst selectivity. Accordingly, disclosed herein is a technique that utilizes a model and reference data specific to the high-selectivity epoxidation catalyst and obtained during offline testing or from historical EO plant operation to account for the various potentially fluctuating operational parameters to determine accurately and reliably, in real-time, an optimal moderator level (Mₒₚₜ) that achieves maximum catalyst selectivity (Sₒₚₜ).

In particular, the present invention is generally directed towards a robust system and method that accurately and reliably determines, in real-time, the optimum catalyst moderator levels that maximize catalyst selectivity in the presence of operational changes of an EO production system and provides actionable guidance to adjust the moderator level such that the catalyst performance is optimized. The system and method disclosed herein use information obtained from models and decision trees in combination with empirical historical data generated by the EO production system over time and catalyst specific reference data routinely obtained during catalyst development and support or from historical EO plant operation. Unlike existing moderator optimization techniques, the system and method disclosed herein does not rely on monitoring the gas phase moderator concentration in the reactor or the feed gas, which may be difficult to obtain reliably and may not be representative of a concentration of the catalyst moderator on the surface of the catalyst. Rather, the disclosed system and method relies on the overall impact of the catalyst moderator (i.e., on a surface of the catalyst) on catalyst performance. Additionally, the method provides a specific magnitude of the moderator level change required to reach optimum rather than only providing directional advice. For example, by using a model, variations from differences in operating conditions (e.g., EO production parameter, reactor feed gas composition, pressure or gas hourly space velocity) that may otherwise confound determination of the optimal catalyst moderator level are removed.

As discussed above, certain ethylene epoxidation catalysts are subject to aging-related performance decline during normal operation of systems used for generating ethylene oxide (EO). Catalyst aging is observed by a reduction in activity and selectivity of the catalyst over time. Therefore, to compensate for the reduced catalyst activity, a temperature within a reactor in which epoxidation takes place is increased. The temperature within the reactor is changed over time with catalyst aging. Even for a given catalyst age, the temperature requirement also changes when conditions such as the desired EO production parameter or feed composition changes. In plant operation, both the decline-related changes in temperature and those related to operating condition changes are common, and it is advantageous to account for both types of changes in any scheme to optimize the moderator level. Incorporating the effects of catalyst aging and changes in operating conditions over longer periods of operating time improves accuracy compared to existing techniques. The present invention accounts for both types of changes to determine accurately and reliably, in real-time, an optimal moderator level (Mₒₚₜ) that achieves maximum catalyst selectivity (Sₒₚₜ) at any catalyst age or operating condition.

### Reference Curves

The optimal moderator level (Mₒₚₜ) depends on the epoxidation reaction conditions and the type of catalyst used. High-selectivity epoxidation catalysts, such as silver-based catalysts having a promoting amount of rhenium (Re), display a maximum selectivity (Sₒₚₜ) at a specific moderator level (Mₒₚₜ). As such, the curves representing the catalyst selectivity as a function of moderator level have a complex shape that includes a maximum in selectivity indicating that the selectivity varies considerably with relatively small changes in the catalyst moderator level, as disclosed in EP 0352850 A1.

The behavior of the high-selectivity epoxidation catalyst under various reaction conditions may be determined from reference curves generated using offline testing data that are routinely gathered during catalyst development and evaluation or from EO plant operation. These reference curves may be used to identify moderator levels that result in maximum catalyst selectivity. For example, using the offline testing data, reference curves that define a catalyst performance relationship to a moderator level may be obtained. These reference curves may be used in combination with the techniques disclosed herein to efficiently and effectively determine the relative effective moderator level (RCl_{eff}) of the catalyst in real-time during operation of the EO production system.

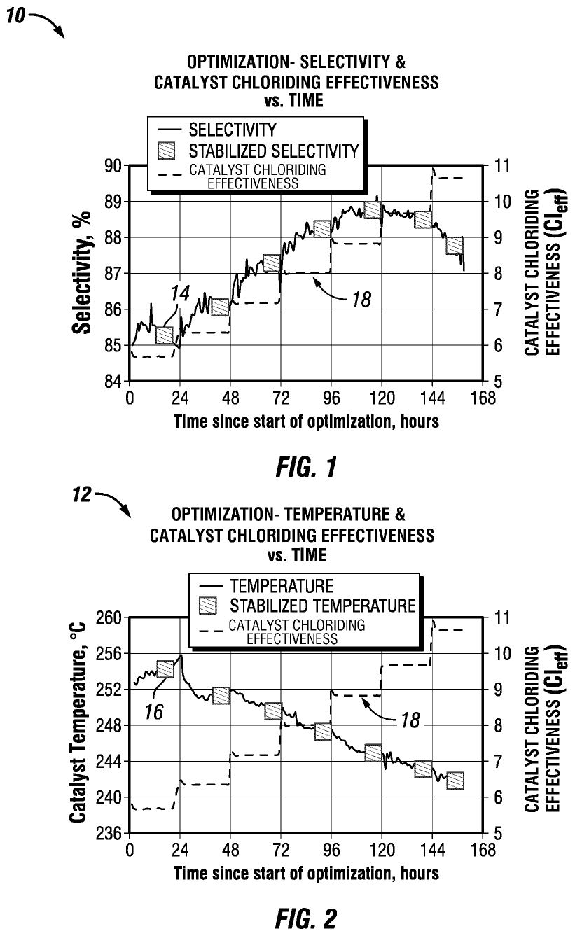

To facilitate discussion of the present invention, the following is a brief description of how the reference curves used in accordance with the disclosed embodiments are obtained. FIGS. 1 and 2 illustrate plots 10 and 12 showing the response of catalyst selectivity (%) and temperature (° C.), respectively, as moderator levels are changed over time for an EO production system at constant operating conditions and a constant EO production parameter. The plots 10 and 12 were generated using microreactor data obtained during optimization of a high-selectivity epoxidation catalyst (i.e., a silver-based catalyst having a rhenium promotor). While the plots 10 and 12 were generated using data from microreactor testing, it should be appreciated that the data may also be generated in a commercial EO plant. As shown in FIGS. 1 and 2, the moderator level (i.e., catalyst chloriding effectiveness value, Cl_{eff}) was stepped manually approximately once per day. A period of time was allowed for the effects of each moderator step on the selectivity of the catalyst to stabilize. Once stabilized, stabilized data points 14 and 16 for the selectivity and the temperature, respectively, were extracted at each moderator level 18.

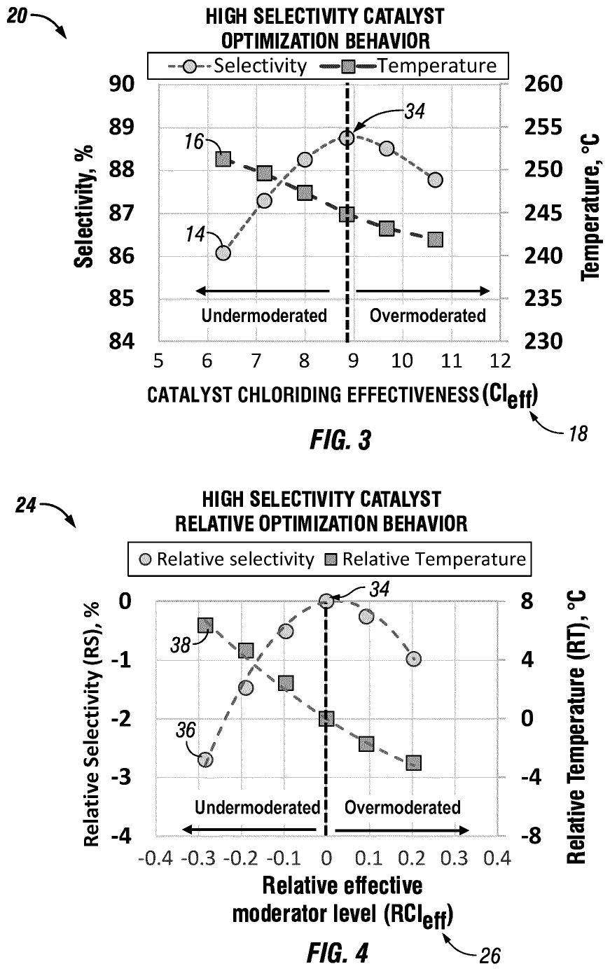

The extracted stabilized data points 14 and 16 may be used to generate an additional plot for the catalyst selectivity and the temperature as a function of the moderator level 18. For example, FIG. 3 illustrates a plot 20 of the selectivity and the temperature as a function of the moderator level 18 generated using the respective extracted stabilized data points 14 and 16. The plot 20 shows a trend in the behavior of a high-selectivity epoxidation catalyst as the moderator level is changed. For example, as the moderator level is increased at a constant target EO production parameter, the selectivity of the catalyst (e.g., data points 14) goes through a maximum point 34, and the temperature (e.g., data points 16) drops. In addition, from the plot 20, performance in which the moderator level is at optimum (e.g., at or near the selectivity maximum point 34), above optimum (i.e., overmoderated) and below optimum (i.e., undermoderated) are shown. In the illustrated plot 20, when the extracted stabilized data points 14 and 16 are on the left of the maximum point 34, the catalyst is in an undermoderated state and the moderator level may be increased to improve the selectivity of the catalyst. Conversely, when the extracted stabilized data points 14 and 16 are on the right of the maximum point 34, the catalyst is in an overmoderated state and the moderator level may be decreased to improve the selectivity of the catalyst.

An alternative representation of the extracted stabilized data points 14 and 16 for selectivity and temperature, respectively, that centers the trends around the maximum point 34 is shown in the plot 24 of FIG. 4. Data points 36 and 38 for relative selectivity (RS) and relative temperature (RT), respectively, in plot 24 are generated by taking the difference between the extracted stabilized selectivity and temperature data points 14 and 16 and the values of selectivity and temperature at the optimum moderator level (Mₒₚₜ) using EQ. 6 and EQ. 7, respectively. In this particular plot, the respective data points 36 and 38 are plotted as a function of the relative effective moderator level 26 (RCl_{eff}) obtained using EQ. 3. The RCl_{eff} 26 indicates the degree that the catalyst is undermoderated or overmoderated. For example, an RCl_{eff}=−0.2 indicates a moderation state that is 20% undermoderated and an RCl_{eff}=+0.15 indicates a moderation state that is 15% overmoderated.

The selectivity curves associated with high-selectivity epoxidation catalysts illustrated in FIGS. 3 and 4 have a pronounced maximum (e.g., the maximum point 34) that is indicative of the optimum catalyst moderator level that achieves the maximum catalyst selectivity for a given set of reaction conditions. In addition, the amount of moderator required to achieve maximum selectivity typically changes as a function of the temperature as the catalyst ages or operating conditions change. While temperature largely contributes to changes in the optimum moderator level (Mₒₚₜ), other factors such as the EO production parameter, the feed gas composition, and other operating conditions result in changes in the optimum moderator concentration as well. For example, as the work rate or reactor inlet CO₂ level changes, the optimum moderator level (Mₒₚₜ) also changes. Accordingly, the moderator concentration may need to be adjusted (i.e., increased or decreased) to maintain maximum catalyst selectivity as these conditions change.

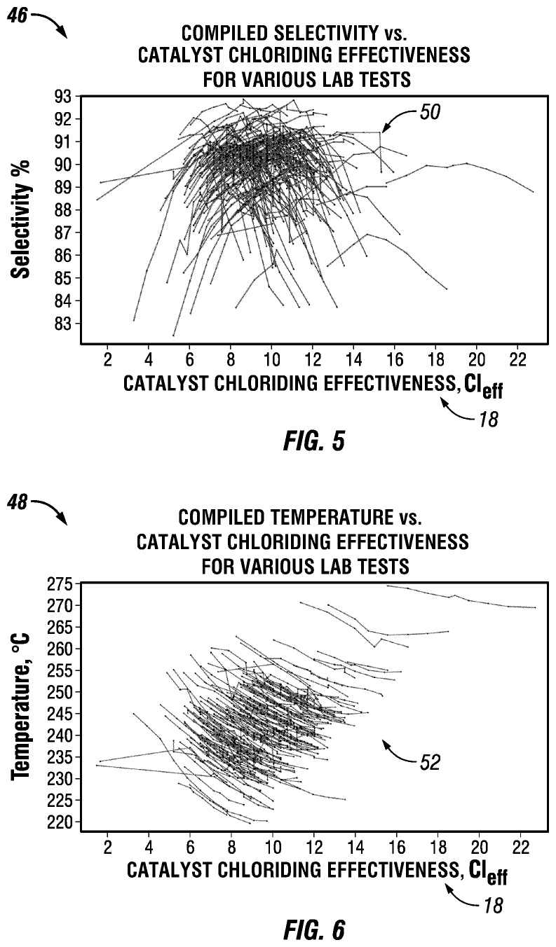

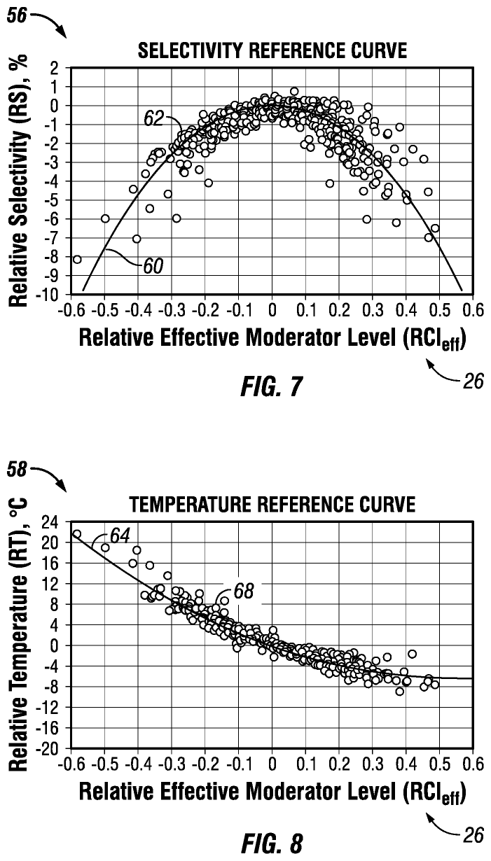

FIGS. 1-4 depict the behavior of a single optimization example for a given catalyst type, age and set of operating conditions. However, similar curves may be generated from data collected in a laboratory during catalyst development and assessment for a variety of different catalyst ages and different operating conditions. For example, FIGS. 5 and 6 illustrate plots 46 and 48 of the selectivity and the temperature, respectively, as a function of the catalyst chloriding effectiveness (Cl_{eff}) generated by compilations of respective data 50 and 52 from various tests associated with a given catalyst at different ages and a wide range of operating conditions such as EO production parameter, GHSV, 02 feed gas concentration, C₂H₄ feed concentration, CO₂ feed concentration, and reactor inlet pressure. As shown in the respective plots 46 and 48, the selectivity, temperature, and values of catalyst chloriding effectiveness vary over a very wide range (e.g., >10% selectivity, >50° C., and a factor of 10× in the catalyst chloriding effectiveness). However, surprisingly, if these data 50 and 52 are replotted in relative terms (e.g., RS, RT, and RCl_{eff}), the wide range of performance data collapses to a much tighter range, as shown in FIGS. 7 and 8.

For example, FIGS. 7 and 8 are representative reference plots 56 and 58 of the relative selectivity (RS) deviation from optimum in percent (%) and relative temperature (RT) compared to the optimum point 34 in degrees Celsius (° C.) calculated from EQS. 6 and 7, respectively, as a function of the RCl_{eff} 26 calculated from EQ. 3 for the various offline laboratory tests at different conditions, as shown in FIGS. 5 and 6. By definition, RS and RT are both 0.0 at the optimum RCl_{eff}, which is also 0.0. As illustrated, a fitted selectivity reference curve 60 and a fitted temperature reference curve 64 may represent the wide range of experimental data 62 and 68, respectively, examined. The fitted reference curves 60 and 64 may be determined using a wide range of readily available statistical curve fitting techniques known to a person skilled in the art, constrained to go through the point (0.0, 0.0).

Although a wide variety of laboratory data 50 and 52 was collected as shown in FIGS. 5 and 6 to generate the representative reference plots 56 and 58 of FIGS. 7 and 8, it will be appreciated that this is not necessary to practice the method disclosed herein. Because selectivity and temperature data collected over a wide variety of operating conditions and catalyst ages surprisingly collapse onto a fitted selectivity reference curve and a fitted temperature reference curve when plotted in relative terms (RS and RT vs. RCl_{eff}), it is not necessary to generate such a wide set of data in a laboratory during catalyst development in order to arrive at the representative reference plots. For example, an EO plant operator may operate the EO plant early in the catalyst run at a particular set of operating conditions in such a way as to collect a single set of selectivity and temperature data over a range of moderator levels that encompass undermoderation, optimum moderation and overmoderation. The EO plant operator may use these data to generate the representative reference plots for use during subsequent operation of the catalyst.

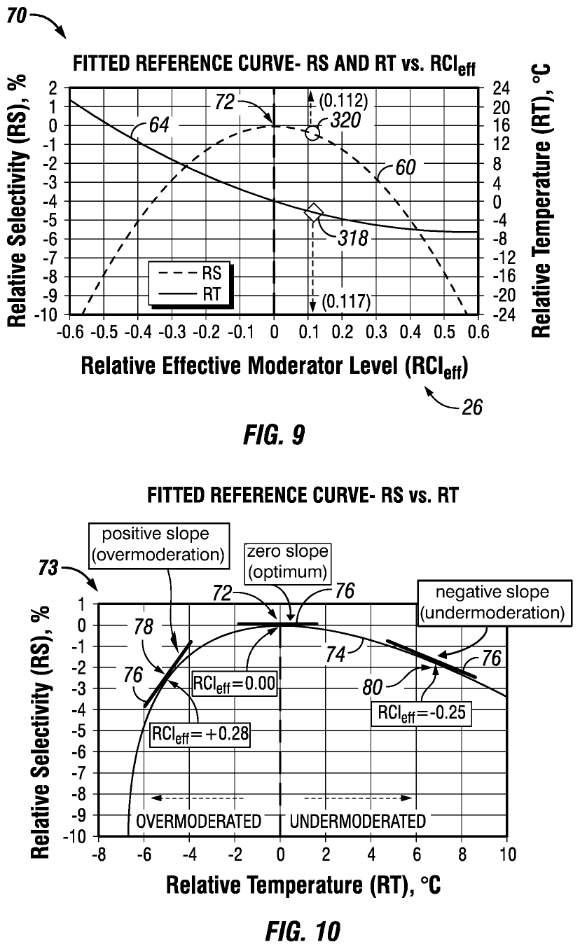

FIG. 9 is a reference plot 70 that replots the fitted reference curves 60 and 64 shown in FIG. 7 and FIG. 8 without all of the underlying data points to show the relevant trends in a single plot. The reference plot 70 relates a given relative selectivity (RS) and relative temperature (RT) to the RCl_{eff} 26. In this plot, by definition, a maximum 72 of the fitted selectivity curve 60 occurs at the point where the RCl_{eff} is zero. As discussed in further detail below, these curves may be used as a helpful reference for optimizing the moderator level in real-time operation of the EO production system.

An alternative view of the fitted reference curves 60 and 64 is shown in FIG. 10, which illustrates a plot 73 of the data as RS vs. RT. In this view, data points to the left of the maximum 72 are overmoderated and the data points to the right of the maximum 72 are undermoderated. Each data point along a curve 74 has a value of RCl_{eff} associated with it, as derived from FIG. 9. Examination of a slope 76 of the fitted curve 74 in this representation may provide valuable insights into the state of the catalyst moderation. For data points to the left of the maximum 72 such as data point 78, the slope 76 of the line is positive, which corresponds to overmoderation. For data points to the right of the maximum 72 such as data point 80, the slope 76 of the line is negative, which corresponds to undermoderation. If the data point is near the maximum 72, the slope 76 will be near zero, which corresponds to optimum moderation. In this way, the slope 76 of the curve 74 may be used to determine the moderation status of the catalyst. In practice, the slope 76 of the RS vs. RT curve 74 at a given data point can be determined by taking the derivative of the fitted function.

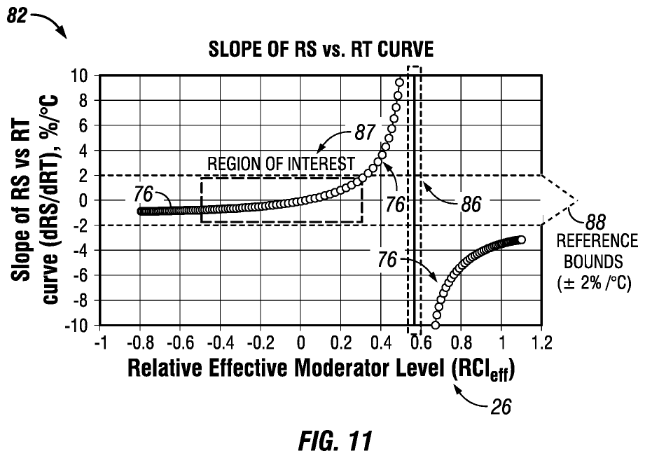

FIG. 11 illustrates a plot 82 of the slope

$$\frac{d ( RS )}{d ( RT )}$$

(e.g., the slope 76) of the RS vs. RT curve 74 as a function of the RCl_{eff} 26 associated with each data point from the curve 74 of FIG. 10, and extending to a broad range of the RCl_{eff} In FIG. 11, a trend of the slope

$$\frac{d ( RS )}{d ( RT )}$$

is shown over a broad range of the RCl_{eff} that extends from −0.8 (80% undermoderated) to 1.1 (110% overmoderated). Over this range, the trend may exhibit a discontinuity 86 where the slope approaches infinity on the overmoderated side. However, the range of interest for optimization during normal plant operation is the range in the general vicinity of the maximum (i.e., the maximum 72), illustrated by a region of interest 87. In general, the range of the slope 76 in the region of interest 87 are within normal reference bounds 88 where there is a unique one-to-one relationship between the slope 76 and the relative effective moderator level 26. For example, in the illustrated plot 82, the normal reference bounds are −2%/° C. to +2%/° C. However, the normal reference bounds may range from ±1%/° C. to ±3%/° C. based on the specific high-selectivity epoxidation catalyst.

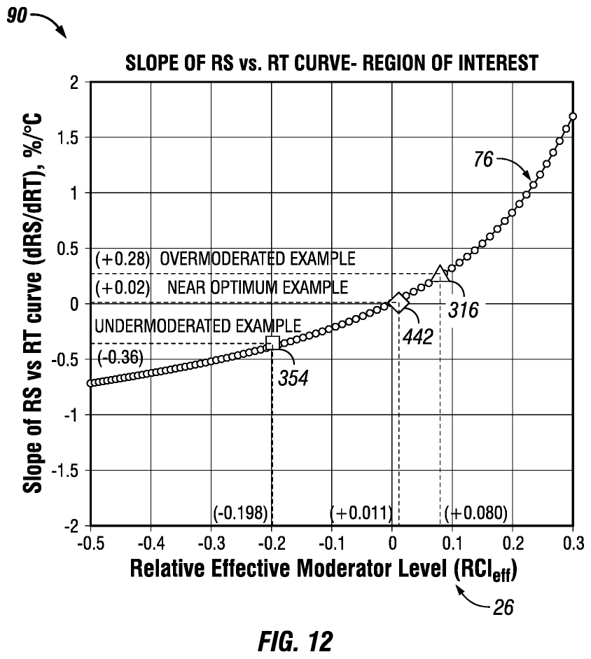

FIG. 12 is a zoomed-in view of the plot of FIG. 11 around point 0, 0 which illustrates a plot 90 that focuses on the slope

$$\frac{d ( RS )}{d ( RT )}$$

of the RS vs. RT curve over the region of interest (e.g., the region 87 in FIG. 11), which is at the RCl_{eff} 26 that is between approximately −0.5 to approximately +0.3. Within this region of interest, there is a one-to-one relationship between the slope

$$\frac{d ( RS )}{d ( RT )}$$

and the RCl_{eff} 26. In practice, the value of the slope

$$\frac{d ( RS )}{d ( RT )}$$

may be assessed if it lies within the normal reference bounds 88 to determine if the catalyst behavior is in the normal range. If the slope

$$\frac{d ( RS )}{d ( RT )}$$

is outside the normal reference bounds (e.g., >+2%/° C. or <−2%/° C.), then the catalyst may be considered to be very overmoderated at the real-time data point (e.g., more than 30% overmoderated), and appropriate corrective actions may be taken as discussed in further detail below.

The data used to generate the reference curves described above with reference to FIGS. 7-12 may be collected offline during testing of catalysts for development or support. However, as should be appreciated, these same reference curves may also be generated during commercial plant operation. As discussed above, the reference curves may cover the catalyst behavior over a wide range of conditions, and they provide an effective and efficient way for moderator optimization compared to existing techniques. Additional catalyst-specific reference curves for different catalyst types may be utilized and applied as needed.

A particular advantage of the present invention is that rather than requiring a full optimization procedure in the plant during every operation in accordance with the teaching of the prior art, the present invention allows for reference curves generated in advance in laboratory scale or smaller scale pilot plant testing to be utilised in informing on optimization procedures in subsequent larger scale operation. The application of reference curves generated in such testing offers greater efficiencies and lower costs to the plant operator than having to rely upon conducting experimentation during commercial operation, particularly when such experimentation would be required to be reconducted in commercial operation every time that there would be changes in operating conditions or temperatures in the plant. By obtaining reference curves in advance in laboratory scale or smaller scale pilot plant testing for a given catalyst composition, such curves may be applied in future operation of the catalyst composition for larger scale operation across multiple plants.

### EO Production System

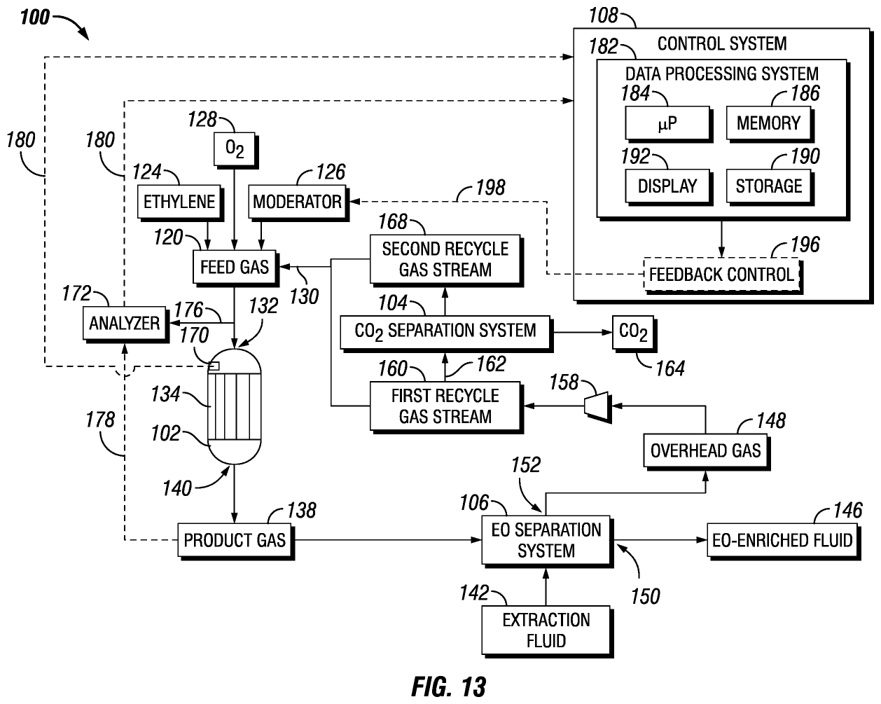

With the foregoing in mind, FIG. 13 illustrates an ethylene oxide production system 100 that may utilize the disclosed process to determine an optimal moderator level for maximum catalyst selectivity in real-time, in accordance with an embodiment of the present invention. In the illustrated embodiment, the system 100 includes an epoxidation reactor system 102, a carbon dioxide (CO₂) separation system 104, an ethylene oxide (EO) separation system 106 and a control system 108. The epoxidation reactor system 102 may include one or more reactors in parallel or in series. In operation, the epoxidation reactor system 102 receives a feed gas 120 that includes ethylene 124, a catalyst moderator 126, oxygen (O₂) 128 and a recycle mixed gas stream 130. The catalyst moderator 126 (e.g., chloride-containing moderator) may include, but is not limited to, C₁ to C₃ chlorohydrocarbons, such as methyl chloride, ethyl chloride, ethylene dichloride, vinyl chloride, and combinations thereof.

As illustrated in FIG. 13, the feed gas 120 is fed to the epoxidation reactor system 102 via a reactor inlet 132. In the epoxidation reactor system 102, the ethylene 124 and oxygen 128 in the feed gas 120 react in the presence of an epoxidation catalyst 134 to generate a product gas 138. The product gas 138 is a mixture of EO, unreacted ethylene 124 and oxygen 128, the catalyst moderator 126, various by-products of the epoxidation reaction (i.e., CO₂ and water (H₂O)), diluent gases and other impurities. The epoxidation process in the reactor system 102 disclosed herein may be carried out under a broad range of operating conditions that may vary widely between different ethylene oxide plants using the system 100 depending, at least in part, upon the initial plant design, subsequent expansion projects, feedstock availability, the type of catalyst used, process economics, etc. Examples of such operating conditions include, but are not limited to, reactor inlet pressure, gas flow through the reactor system 102 (commonly expressed as the gas hourly space velocity or “GHSV”), feed gas composition, and the ethylene oxide production parameter (commonly described in terms of work rate).

To achieve desired commercial ethylene oxide production rates, the epoxidation reaction is typically carried out at a reaction temperature of approximately 180° C. or higher, or approximately 190° C. or higher, or approximately 200° C. or higher, or approximately 210° C. or higher, or approximately 225° C. or higher. Similarly, the reaction temperature is typically approximately 325° C. or lower, or approximately 310° C. or lower, or approximately 300° C. or lower, or approximately 280° C. or lower, or approximately 260° C. or lower. The reaction temperature may be from approximately 180° C. to 325° C., or from approximately 190° C. to 300° C., or from approximately 210° C. to 300° C. It should be noted that the term “reaction temperature” as used herein refers to any selected temperature(s) that are directly or indirectly indicative of the catalyst bed temperature. For example, the reaction temperature may be a catalyst bed temperature at a specific location in the catalyst bed or a numerical average of several catalyst bed temperature measurements made along one or more catalyst bed dimensions (e.g., along the length). Alternatively, the reaction temperature may be, for example, the gas temperature at a specific location in the catalyst bed, a numerical average of several gas temperature measurements made along one or more catalyst bed dimensions, the gas temperature as measured at the outlet of the epoxidation reactor, a numerical average of several coolant temperature measurements made along one or more catalyst bed dimensions, or the coolant temperature as measured at the inlet or outlet of the epoxidation reactor, or in the coolant circulation loop. One example of a well-known device used to measure the reaction temperature is a thermocouple.

The epoxidation processes disclosed herein are typically carried out at a reactor inlet pressure of from approximately 1000 to 3000 kPa, or from approximately 1200 to 2500 kPa, absolute. A variety of well-known devices may be used to measure the reactor inlet pressure, for example, pressure-indicating transducers, gauges, etc., may be employed. It is within the ability of one skilled in the art to select a suitable reactor inlet pressure, taking into consideration, for example, the specific type of epoxidation reactor, desired productivity, etc.

The gas flow through the epoxidation reactor is expressed in terms of the Gas Hourly Space Velocity (“GHSV”), which is the quotient of the volumetric flow rate of the feed gas 120 at standard temperature and pressure (i.e., 0° C., 1 atm) divided by the catalyst bed volume (i.e., the volume of the reactor system 102 that contains the epoxidation catalyst 134). The GHSV represents how many times per hour the feed gas 120 would displace the catalyst volume in the reactor system 102 if the feed gas 120 were at standard temperature and pressure (i.e., 0° C., 1 atm). Typically, in a gas phase epoxidation process, the GHSV is from approximately 1,500 to 10,000 per hour.

As previously discussed, the production rate of ethylene oxide in the reactor system 102 is typically described in terms of an EO production parameter such as work rate, which refers to the amount of ethylene oxide produced per hour per unit volume of catalyst. In general, for a given set of operating conditions, increasing the reaction temperature at those conditions increases the work rate, resulting in increased ethylene oxide production. However, this increase in temperature often reduces catalyst selectivity and may accelerate the aging of the catalyst. Alternatively, as an epoxidation catalyst undergoes natural catalyst aging over time, the work rate will start to naturally decrease for a given reaction temperature. Under such circumstances, the reaction temperature is increased in order to maintain work rate at the required value. Typically, the work rate in most plants is from approximately 50 to 400 kg of ethylene oxide per m³ of catalyst per hour (kg/m³/h), or from approximately 120 to 350 kg/m³/h. One skilled in the art with the benefit of the present invention will be able to select appropriate operating conditions, such as the feed gas composition, reactor inlet pressure, GHSV, and work rate depending upon, for example, plant design, equipment constraints, the age of the epoxidation catalyst, etc.

As discussed above, the reactor system 102 generates the product gas 138, which is a mixture of EO, unreacted ethylene 124 and oxygen 128, the catalyst moderator 126, various by-products of the epoxidation reaction (i.e., CO₂ and water (H₂O)), diluents and other impurities. The product gas 138 exits the epoxidation reactor system 102 via reactor outlet 140 and is fed to the EO separation system 106. In the EO separation system 106, the EO is separated from the product gas 138 via any suitable separation technique. For example, in the illustrated embodiment, an extraction fluid 142 such as water may be used to separate the EO from the product gas 138. The extraction fluid 142 removes the EO from the product gas 138 to generate an EO-enriched fluid 146 having the EO. The EO-enriched fluid 146 exits the EO separation system 106 through a first outlet 150 (e.g., EO outlet) and may be further processed and used to provide products such as glycols (e.g., ethylene glycol, diethylene glycol, triethylene glycol, etc.) via catalytic or non-catalytic hydrolysis. An overhead gas 148 that includes unreacted ethylene 124 and oxygen 128, by-products (CO₂ and H₂O) and other diluents and impurities exits the EO separation system 106 through a second outlet 152 and is recycled to the reactor system 102 (e.g., via recycle gas stream 130). A compressor 158 or other suitable device may be used to facilitate transport of the overhead gas 148 through the system 100. In the illustrated embodiment, a first recycle gas stream 160 exiting the compressor 158 is directed to the feed gas 120. A portion 162 of the first recycle gas stream 160 is fed to the CO₂ separation system 104 where CO₂ is separated from the first recycle gas stream 160 to generate CO₂ 164 and a second recycle gas stream 168. The second recycle gas stream 168 is combined with the first recycle gas stream 160 to generate the recycle mixed gas stream 130, which is combined with a fresh feed of ethylene 124, the catalyst moderator 126, and the oxygen (O₂) 128 to form feed gas 120 and fed to the reactor system 102. In this way, the unreacted ethylene and oxygen in the product gas 138 may be fed back to the reactor system 102, thereby improving the overall efficiency of the EO production system 100.

As discussed above, the catalyst moderator (e.g., the moderator 126) plays an important role in maintaining the activity and selectivity of the catalyst (e.g., the epoxidation catalyst 134) used for the production of EO. When using high-selectivity epoxidation catalysts such as silver-based catalysts having a promoting amount of rhenium, maximum selectivity of the catalyst is obtained within a narrow moderator level in the feed gas (e.g., the feed gas 120). However, the optimum moderator level (Mₒₚₜ) at which the catalyst selectivity is at maximum (Sₒₚₜ) is not fixed and varies based on the reaction temperature and the operating conditions. As the performance of the catalyst declines over time, the reaction temperature is increased to improve performance of the catalyst and maintain a constant rate of EO production. Therefore, the level of moderator 126 in the feed gas 120 is typically adjusted along with the reaction temperature and the reaction operating conditions such as feed concentrations or the EO production parameter (e.g., work rate) to maintain the catalyst 134 operating at maximum selectivity (S opt). As discussed in further detail below, the techniques disclosed herein use a combination of real-time data, model data, reference data (e.g., the reference data in FIGS. 7-12) and empirical historical data to determine an optimum moderator level (Mₒₚₜ) that achieves maximum catalyst selectivity (Sₒₚₜ). The techniques also provide actionable guidance for adjusting the level of the moderator 126 to the optimum moderator level (Mₒₚₜ) in real-time with improved accuracy and reliability compared to existing techniques.

The EO production system 100 in FIG. 13 includes one or more sensors 170 and/or an analyzer system 172 that measure and monitor one or more operating parameters of the system 100 in real-time. The analyzer system 172 may include one or more analyzers that analyze the feed gas 120, the product gas 138 or both. For example, in operation, the analyzer system 172 receives a portion 176 of the feed gas 120 and measures a concentration of the moderator 126 (i.e., chlorides) and other components in the feed gas 120. In certain embodiments, the analyzer system 172 may receive a portion 178 of the product gas 138 and measure a concentration of EO and other components in the product gas 138. The analyzer system 172 may include a gas chromatograph (GC), a mass spectrometer (MS) or any other suitable analytical tool for analyzing the feed gas 120 and/or the product gas 138 and combinations thereof. In the illustrated embodiment, one or more sensors 170 are positioned within the reactor system 102 and may monitor a temperature of a coolant supplied to the reactor 102 and/or reacting gas temperatures at one or more locations along the reactor 102. The sensors 170 may be positioned within a shell of the reactor or reactors, in the reactor or reactors themselves, in a coolant circulation loop, within select catalyst tubes and combinations thereof. In certain embodiments, the sensors 170 may be positioned at the reactor outlet 140. As such, the sensors 170 may measure and monitor a temperature of the product gas 138 exiting the reactor 102. The temperature of the product gas 138 may also provide insight as to the temperature within the reactor system 102 and, consequently, the activity of the catalyst 134.

### Control System

The data collected, in real-time, from the sensors 170 and the analyzer 172 may be used to determine the performance of the catalyst 134 and whether the level of the moderator 126 is at or near optimum for maximum catalyst selectivity (Sₒₚₜ). Accordingly, in the illustrated embodiment, the sensors 170 and the analyzer 172 transmit real-time data 180 to the control system 108 where the data 180 may be stored and processed in a data processing system 182. The control system 108 may receive the real-time data 180 via a wired or wireless connection. In certain embodiments, the control system 108 is located in a remote location that is separate from the EO plant. The data 180 may include a plurality of measurements (e.g., chloride concentration in the feed gas 120 or rate of addition of the moderator 126, the EO concentration in the product gas 138, the measured selectivity, the reaction temperature, pressure, gas hourly space velocity (GHSV), feed gas composition, EO production parameter, etc.) associated with the operation of the reactor system 102. The data processing system 182 may use the data 180 to determine, in real-time, the optimum level of the moderator (Mₒₚₜ) 126 that maximizes catalyst selectivity (Sₒₚₜ) at the operating conditions of the reactor system 102 without requiring an operator to perform several manual steps in the level of the moderator 126 in the reactor system 102. In addition, as discussed in further detail below, the data processing system 182 advantageously determines the optimum moderator level (Mₒₚₜ) and maximum selectivity (Sₒₚₜ) of the catalyst 134 without relying on accurate and precise monitoring of the concentration of the moderator 126 in the feed gas 120 and/or on the surface of the catalyst 134. In certain embodiments, when reliable moderator measurements are available, these measurements may be used in combination with the method disclosed herein to determine the optimum moderator level (Mₒₚₜ).

The data processing system 182 may include a microprocessor (μP) 184, memory 186, storage 190 and/or display 192. The memory 186 may include one or more tangible, non-transitory, machine readable media collectively storing one or more sets of instructions for operating the system 100, estimating the optimum moderator level (Mₒₚₜ) for maximum catalyst selectivity (Sₒₚₜ), determining maximum catalyst selectivity, determining the relative effective moderator level of the catalyst, triggering an alert associated with the catalyst performance and providing actionable guidance/recommendation for a target change that may include adjusting the moderator level to achieve maximum catalyst selectivity. In certain embodiments, the one or more sets of instructions may instruct the system 100 to adjust the level of the moderator 126 (e.g., total weighted moderator concentration, make-up moderator feed rate or catalyst chloriding effectiveness value (i.e., Cl_{eff})) based on the recommendation. For example, in certain embodiments, the control system 108 includes a feedback control element 196 that may receive instructions to adjust automatically the moderator concentration or the feed rate of the moderator 126. The feedback control element 196 may send a signal 198 to a valve that controls a flow of the moderator 126, thereby adjusting the amount of the moderator 126 in the feed gas 120.

The memory 186 may include instructions to predict optimized performance of the catalyst 134 by using a model that accounts for changes in operating parameters of the system 100. Advantageously, the model does not require the level of moderator 126 in the feed 120 and/or on the surface of the catalyst. That is, the model estimates the impacts of changes in pressure, gas hourly space velocity, EO production parameter, feed gas composition, and optionally catalyst age on the catalyst selectivity and temperature for an optimum moderator level (Mₒₚₜ). Accordingly, deviations of the selectivity and temperature of the catalyst relative to the model estimate are related to changes in the relative effective moderator level. The model may be any suitable model that predicts the impact of feed gas composition (e.g., O₂, C₂H₄, CO₂, C₂H₆, CH₄, H₂O), GHSV, pressure and EO production parameter on the chloride-optimized selectivity and temperature, preferably as a function of catalyst age. The model is specific to the catalyst 134 and may be provided by a catalyst supplier. Therefore, in one embodiment, the model is supplied by the catalyst supplier that provided the catalyst, and the model is incorporated into the system and method disclosed herein. In another embodiment, the operator of the catalyst 134 in the EO production system 100 may develop the model based on data collected during process operation. By way of a non-limiting example, the model may be an empirical statistical model, multivariate model, kinetic model, a neural network, or any other suitable model that captures the impact of the operating conditions of the system 100, the catalyst 134 and, preferably, catalyst age. Examples of such models can be found in “An Experimental Study of the Kinetics of Selective Oxidation of Ethene over a Silver on α-Alumina Catalyst”, P C Borman and K R Westerterp, Ind. Eng. Chem. Res, 1995, 34, 49-58 and “Hybrid modeling of ethylene to ethylene oxide heterogeneous reactor”, G Zahedi, A Lohi, K A Mandi, Fuel Processing Technology, 2011, 92, 1725-1732. WO 2016/108975 A1 demonstrates an example of a catalyst model and how to generate it in Experiment 1, paragraphs [0077] to [0080]. In certain embodiments, continuous online model refitting techniques may be used. By way of a non-limiting example, continuous online model refitting techniques may include weighted re-estimation of model parameters or other suitable refitting techniques understood by a person having ordinary skill in the art. As should be appreciated, these techniques can be applied by those skilled in the art through collection and fitting of performance data for the high-selectivity epoxidation catalyst of interest.

The memory 186 may store the model, decision trees and any other information that may be used to determine the relative effective moderator level of the catalyst and the moderator-optimized performance of the catalyst as well as provide actionable guidance/recommendations regarding the direction and amount the moderator level should be adjusted to achieve maximum catalyst selectivity. The memory 186 may also store instructions to generate a visualization 192 to display for an operator of the system 100 related to catalyst performance, moderator levels, system performance, and system maintenance. Visualizations include, but are not limited to, plots, data confidence levels, alerts, recommendations, measurements and system parameters among others.

To process the data 180, the processor 184 may execute instructions stored in the memory 186 and/or storage 190. For example, the instructions may cause the processor 184 to estimate changes in the moderator level (M) as a function of changes in temperature (i.e., reaction temperature) or feed composition, compare the observed moderator level to the optimum moderator level (Mₒₚₜ) and reference curves, and determine the impact of the moderator level on catalyst performance without relying on the concentration of the moderator in the feed gas 120. The present invention relies on being able to change effectively the rate of chloride (i.e., moderator) addition, which is a universal requirement for operation of the system 100 and easier than having to monitor the chloride concentration accurately in the feed gas 120. However, to the extent that an accurate and reliable concentration of the chloride in the feed gas 120 is measured, this value may be used to determine the impact of the moderator concentration on the catalyst selectivity. In certain embodiments, the instructions may cause the processor 184 to apply a data pre-processing/cleaning step to average or smooth the data, remove outliers, perform data imputation, etc. As such, the memory 186 and/or storage 190 of the data processing system 182 may be any suitable article of manufacture that can store the instructions. By way of a non-limiting example, the memory 186 and/or storage 190 may be read-only memory (ROM), random-access memory (RAM), flash memory, an optical storage medium, a hard disk drive, cloud storage or other storage media.

The data processing system 182 may transmit data (e.g., measurements, plots, operation parameters, operation conditions, etc.) to external devices (e.g., remote displays, cell phones, tablets, laptops, electronic data management systems, etc.) that may be readily accessible to the EO plant operator. The display 192 may be any suitable local or remote electronic display that can display information (e.g., catalyst selectivity plots, temperature plots, work rate plots, moderator concentration plots, moderator optimization guidance plots, alerts, recommendations, confidence levels, system parameters or any other suitable information and combinations thereof) relating to operation of the system 100. In certain embodiments, the data processing system 182 may use information obtained from modeling operations, ad hoc assertions from the operator, empirical historical data (e.g., historical operating data), and reference data (e.g., data generated during catalyst development and testing) in combination with the data 180 (e.g., real-time data) to determine the optimum moderator level (Mₒₚₜ) for maximum catalyst selectivity (Sₒₚₜ). In one embodiment, the data processing system 182 may retrieve data (e.g., reference data, empirical historical data, etc.) stored on the cloud, servers and/or third party systems to which the data processing system 182 has been granted access.

As discussed above, the data 180 from the system 100 may be analyzed in combination with a model to determine the relative effective moderator level (RCl_{eff}) and performance of the catalyst 134 without relying on precise monitoring of the moderator concentration in the feed gas 120. For example, the model may determine variations in the estimated chloride-optimized selectivity and temperature of the catalyst as the operating conditions (e.g., EO production parameter, pressure, gas hourly space velocity (GHSV), feed composition, etc.) of the system 100 change. The model may also determine long-term decline or catalyst age effects. As discussed in further detail below, at least a portion of the data 180 in combination with the model data and the reference data stored in the data processing system 182 are used to determine the impact of the level of the moderator 126 on the selectivity and activity of the catalyst. As discussed above, the catalyst surface concentration (coverage) of the moderator 126 impacts catalyst performance. However, measuring the concentration of the moderator 126 on the surface of the catalyst is not practical. Existing techniques rely on the measurement of the concentration of the moderator 126 in the feed gas 120, which does not always represent the concentration of the moderator 126 on the surface of the catalyst 134. Accordingly, by not relying on the measured concentration of the moderator 126 in the feed gas 120 or on the surface of the catalyst 134, the techniques disclosed herein provide a more robust, reliable and accurate way to determine optimum moderator level (Mₒₚₜ) and maximum catalyst selectivity (Sₒₚₜ) under any given set of operating conditions in real-time compared to existing techniques. Additionally, the disclosed techniques provide actionable guidance/recommendations for adjusting the moderator level to achieve maximum catalyst selectivity (Sₒₚₜ) without having to manually step the moderator level to find its optimum value (Mₒₚₜ).

By using the data 180 collected in real-time, system historical data (i.e., the empirical data), model data and reference data associated with the impact of changes in operational conditions of the system 100 on the optimum moderator concentration and catalyst performance, the moderator optimization techniques disclosed herein may be tailored to specific systems and catalysts. The model disclosed herein may learn from the historical data obtained throughout the operation of the epoxidation system 100 about the operational parameters, in particular the moderator levels, that provide maximum catalyst selectivity. That is, the model may be fine-tuned by using the real-time data 180 collected over time (e.g., days, weeks, months, years) to provide a reliable and accurate estimate of optimum moderator levels (Mₒₚₜ) for maximum catalyst selectivity (Sₒₚₜ). In certain embodiments, the reference data obtained from offline microreactor testing of the desired catalyst during catalyst development and support or from EO plant operation may be part of the historical data the model may use to fine-tune estimates. Therefore, combining the real-time data 180 with the historical data and the reference data (e.g., the reference data in FIGS. 7-12) facilitates determining moderator concentration adjustments that maximize catalyst selectivity for a given system and catalyst. As such, the disclosed system 100 mitigates the complexities of existing moderator/catalyst optimization techniques that rely on monitoring moderator concentrations and provides for a robust system with improved accuracy and reliability for real-time moderator optimization compared to existing techniques.

### Method to Determine Optimum Moderator Level (Mₒₚₜ)

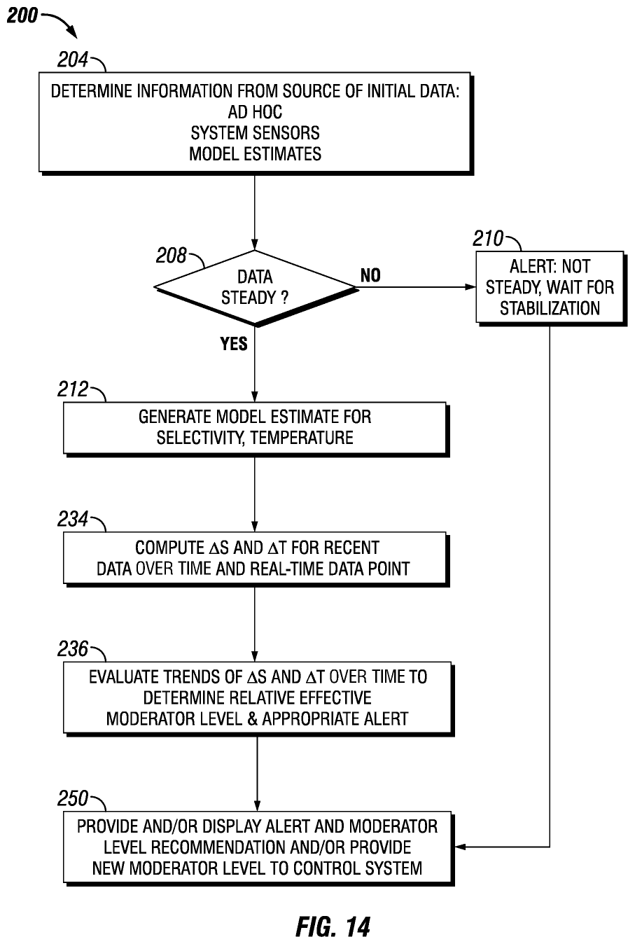

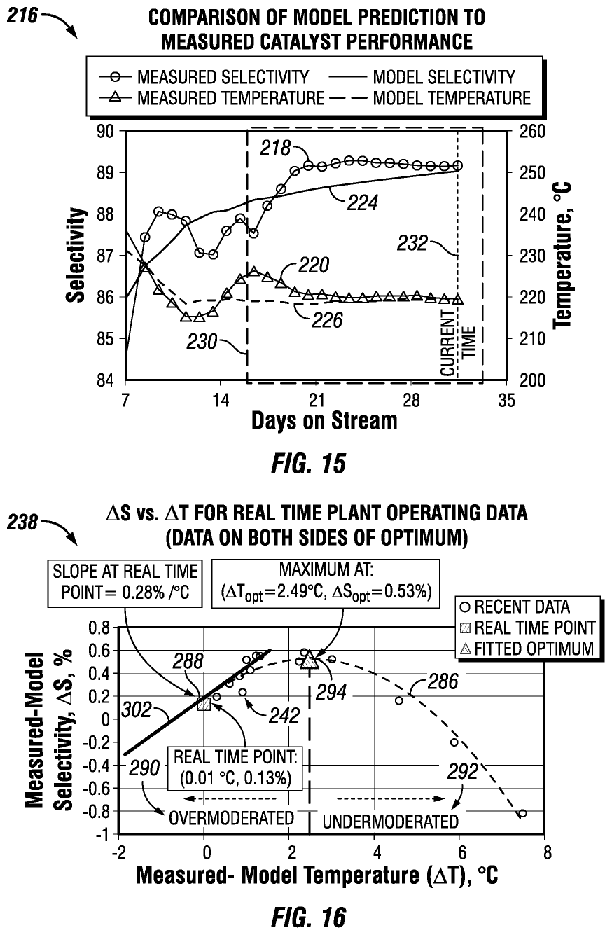

A method 200 for determining the optimum moderator level (Mₒₚₜ) that maximizes catalyst selectivity (Sₒₚₜ) (e.g., the selectivity of the catalyst 134) using the system 100 is illustrated in FIG. 14. To facilitate discussion of certain aspects of the method 200, reference will be made to FIGS. 15-17. In the illustrated method 200, information from sources of initial data may be collected (block 204). The sources of the initial data may include real-time data (e.g., the data 180) and empirical historical data. In certain embodiments, the sources of the initial data may include model data, ad hoc assertions from operators or any other suitable source of information associated with the EO production system. The empirical historical data may include data associated with the EO production system generated over time during operation and/or reference data (e.g., the reference data 62, 68 in FIGS. 7 and 8) obtained during offline catalyst development and support or EO plant operation. The empirical historical data may include catalyst selectivity and temperature responses to changes in the moderator level, feed gas composition, pressure, EO production parameter and gas hourly space velocity among others. The empirical historical data may also include information regarding the moderator levels that maximize catalyst selectivity and the dependence of performance on moderator levels below and above the optimum level for a given set of operating conditions. The data processing system may evaluate the empirical historical data for changes to identify the best analysis parameters and allow tuning for specific systems, thereby improving the reliability and accuracy of the model estimations for maximum catalyst selectivity.

The method 200 also includes a decision step to determine whether the data are steady at query 208. Prior to query 208, the data processing system may pre-process or clean (e.g., statistical cleaning) the data collected in block 204. Techniques such as, but not limited to, data smoothing, anomaly removal and imputation may be used to clean the data. Inclusion of unstable or abnormal conditions outside of the normal operation of the system with excessive deviations in catalyst selectivity and temperature may result in misinterpretation of trends. Therefore, cleaning may remove outliers in the data. Moreover, filtering out data with large condition differences may also improve the extraction of trends and the identification of optimum conditions for catalyst performance. For example, if the current real-time values of certain operating conditions (e.g., EO production parameter, gas hourly space velocity, pressure, etc.) deviate significantly from normal operation, this could indicate potential unstable operation or result in selection of data with variability in conditions that the model may not capture completely. To minimize these effects, the data processing system may apply EO production parameter-based filters, time-based filters or any other suitable filter to select the most relevant data points. Additionally, the data processing system may determine whether all the necessary input data (e.g., EO production parameter, feed composition, GHSV and pressure) are available. If all the necessary input data are not available, the data processing system may apply imputation to estimate the missing input data using any suitable data processing technique. Therefore, pre-processing the data may improve the overall accuracy and reliability of the optimum catalyst moderator levels determined using the system and method disclosed herein.

At query 208, if the data processing system determines that the data are not steady (i.e., unstable), the data processing system provides an alert to wait for data stabilization (block 210). Therefore, data may continue to be collected until the data are stabilized. Conversely, if the data are stabilized, the data processing system proceeds to estimate the model selectivity (Sₑₛₜ), model temperature (Tₑₛₜ), delta selectivity (ΔS) and corresponding delta temperature (ΔT) over time and for a current real-time data point (block 212). For example, as discussed above, the data processing system (e.g., the data processing system 182) stores data collected over time during operation of the EO production system (e.g., the EO production system 100), reference data (e.g., the reference data in FIGS. 7-12) and a model in the storage (e.g., the storage 190). The memory (e.g., the memory 186) stores instructions that when executed by the processor (e.g., the processor 184) retrieve the empirical historical data, real-time data and reference data and generate one or more plots of the catalyst selectivity and temperature over time using the model and the real-time data.

FIG. 15 is a representative plot 216 of catalyst selectivity and temperature (a representation of catalyst activity) as a function of time in days on stream that may be generated in accordance with the acts of block 212. The plot 216 includes measured catalyst selectivity data 218 and measured temperature data 220 collected over time by the EO production system. In addition, the plot 216 includes model-estimated catalyst selectivity (Sₑₛₜ) data 224 and model-estimated temperature (Tₑₛₜ) data 226 over time. The plot 216 may be used to identify deviations in the measured data 218, 220 from the respective model-estimated data 224, 226. Because the model represents moderator-optimized catalyst performance, any deviations of the measured data 218, 220 from the model-estimated data 224, 226, respectively, can be used to extract deviations of the moderator level relative to optimum. Since the method described herein applies deltas between measured and model-estimated selectivities (Sₘₑₐₛ and Sₑₛₜ) and temperatures (Tₘₑₐₛ and Tₑₛₜ), systematic errors in measurement and/or modeling are mitigated and do not affect its applicability. Accordingly, the plot 216 may be used to identify a trend in the data associated with the catalyst performance during operation of the system.

In certain embodiments, the operator of the EO production system may select a section 230 along the plot 216 that is of interest for additional processing. The section 230 may include portions in which the measured data 218, 220 deviated from the respective model-estimated data 224, 226. The measured data 218, 220 and the model-estimated data 224, 226 include measured data and model estimates for selectivity (Sₘₑₐₛ and Sₑₛₜ) and temperature (Tₘₑₐₛ and Tₑₛₜ) for the catalyst run up until a current real-time data point 232, in accordance with block 212 of FIG. 14. As illustrated in FIG. 15, the selected section 230 includes the most recent data collected by the EO production system. The time frame used for the section 230 includes the real-time data point and may be a significant portion, or even the entirety, of the catalyst run. Typical time frames considered for the section 230 may range from 1-2 days to 180 days, depending on the nature of the trends involved and which time frame provides the clearest trend. The choice of time frame to consider for analysis may be much wider than certain existing optimization techniques, such as those described in WO 2016/108975 A1. The broader range of data available to use in the disclosed analysis increases and improves the ability to find an optimum moderator level (Mₒₚₜ) compared to existing techniques.

Returning to FIG. 14, following model-estimation of the selectivity (Sₑₛₜ) and the temperature (Tₑₛₜ) in accordance with block 212, the method 200 includes computing a delta selectivity (ΔS) and a corresponding delta temperature (ΔT) for the recent data and the real-time data point (block 234). For example, the data processing system extracts the measured data (e.g., the measured data 218, 220) and the model-estimated data (e.g., the model-estimated data 224, 226) from the selected section (e.g., the selected section 230) and determines the ΔS and the corresponding ΔT between the respective measured and model-estimated data. The data processing system determines the ΔS by taking the difference between the measured selectivity data (Sₘₑₐₛ) (e.g., the measured data 218) and the model-estimated selectivity data (Sₑₛₜ) (e.g., the model-estimated data 224) for each time point in accordance with EQ. 8, and the ΔT by taking the difference between the measured temperature data (Tₘₑₐₛ) (e.g., the measured data 220) and the model-estimated temperature data (Tₑₛₜ) (e.g., the model-estimated data 226) for each time point in accordance with EQ. 9.

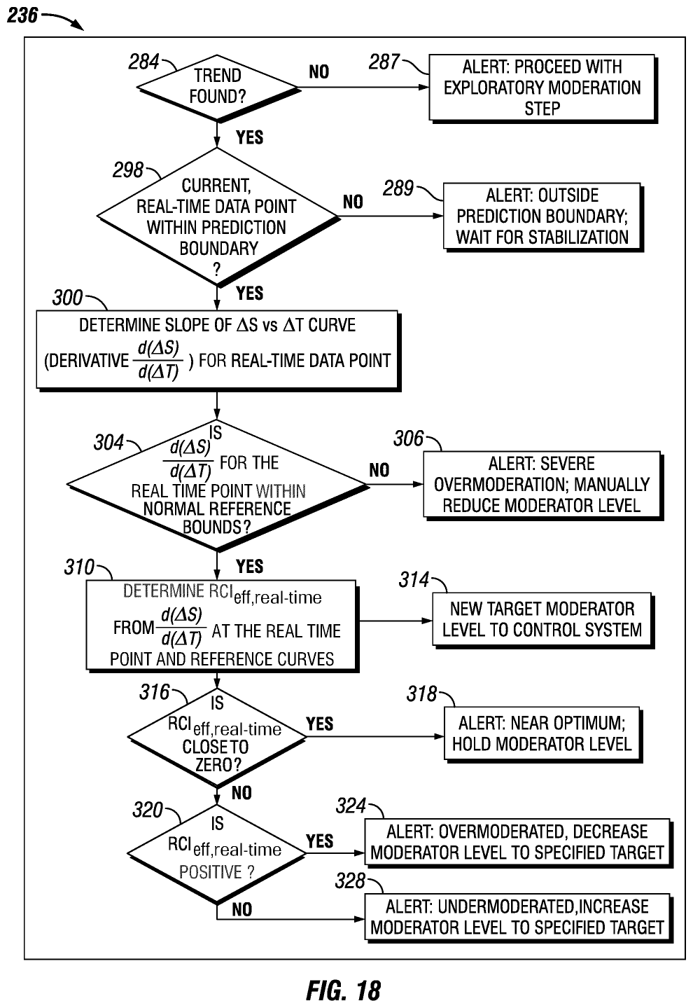

The ΔS and the ΔT values may also be further processed to estimate the optimum moderator level (Mₒₚₜ). Accordingly, the method 200 includes evaluating trends in the ΔS and ΔT values to determine an RCl_{eff} level (block 236). The acts of block 236 include a decision tree having various decision steps for evaluating the trends and determining the RCl_{eff} Accordingly, the step 236 is shown in greater detail as decision tree 236 (FIG. 18). FIG. 16 illustrates a plot 238 of ΔS vs. ΔT for the selected data (e.g., the data in section 230 of FIG. 15) with a fitted curve through the data. The point on the fitted curve which maximizes selectivity (ΔSₒₚₜ) is at the optimum moderator level (Mₒₚₜ). The value of ΔT on the fitted curve for ΔS vs. ΔT corresponding to ΔSₒₚₜ is ΔTₒₚₜ.

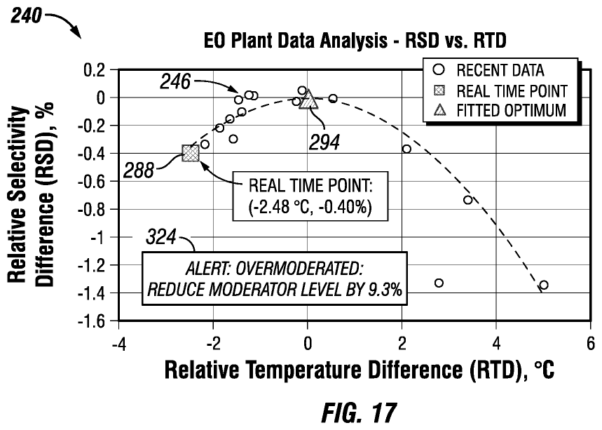

FIG. 17 illustrates a plot 240 that represents the data of FIG. 16 in relative terms (e.g., relative selectivity difference (RSD), relative temperature difference (RTD)) by subtracting the fitted optimum values ΔSₒₚₜ and ΔT op t at point 294 from the respective ΔS and ΔT using EQ. 10 and EQ. 11. Plotting the data as RSD vs. RTD rather than ΔS vs. ΔT simply shifts the resultant curve so that its maximum point is fixed at (0,0) by definition. In the embodiment illustrated in FIG. 16, data set 242 represents the ΔS and ΔT values for individual days of data collected during a selected period of time (e.g., the selected section 230). In the embodiment illustrated in FIG. 17, data set 246 represents the RSD and RTD values for the individual days of data collected during the selected period of time. Another suitable time frequency may also be used for the plots 238, 240 (e.g., hours instead of days).

The model disclosed herein estimates the impact of operating conditions at an optimized moderator level. Therefore the difference between the measured data at those operating conditions and the model-estimated data (i.e., ΔS and ΔT) effectively removes the effect of operating conditions on the catalyst performance, leaving only the impact of the moderator level, as shown in FIGS. 16 and 17. Hence, the trends depicted in FIGS. 16 and 17 are analogous to that of the fitted reference curve shown in FIG. 10. Thus, FIG. 16 may be compared to the reference curve in FIG. 10 to estimate the direction of catalyst moderation (undermoderation or overmoderation). Using the RSD and RTD (e.g., FIG. 17), the magnitude of undermoderation or overmoderation may also be determined. Likewise, the slopes of the curves shown in FIG. 16 or 17 may be used analogously to the reference information in FIG. 12 to determine the RCl_{eff} for a given real-time data point (RCl_{eff}_{ᵣₑₐₗ₋ₜᵢₘₑ}), as discussed in further detail below and illustrated in Examples 1, 4 and 5.

As discussed above, the system and methods disclosed herein also include providing an alert and/or actionable recommendation/guidance based on the moderation level of the EO production system relative to the optimum point determined using a combination of historical operating data, reference data and model data. Accordingly, returning to FIG. 14, the method 200 includes providing and displaying an alert and/or actionable recommendation or a new moderator level to the control system in accordance with block 250. The data processing system may use one or more decision trees (e.g., the decision tree 236) having a set of conditions outlined that, when met, cause the data processing system to output a respective recommendation.

In embodiments in which the catalyst is overmoderated or undermoderated, the data processing system may provide an audio or visual alert indicating that the catalyst performance is not at optimum. By way of non-limiting example, the alert may be an alarm, a notification on a display (e.g., the display 192 or other remote display on a phone, a laptop, a tablet, etc.), activation of a light, a color change in a light or the display (e.g., from green to yellow or green to red), or any other suitable audio or visual alert and combinations thereof that alerts the operator that the system is not operating at optimum conditions. In this way, the operator of the EO production system may be notified/alerted as to whether the moderator level (moderator concentration, catalyst chloriding effectiveness value (Cl_{eff}), or moderator feed rate) needs to be adjusted to improve performance of the catalyst.

In certain embodiments, the data processing system may provide actionable advice to the operator on recommended adjustments (i.e., target changes) to the moderator level to achieve maximum catalyst selectivity. As discussed above, the data processing system may use reference data (e.g., the reference data 62, 68 in FIGS. 7 and 8, respectively) obtained during offline testing of the catalyst or earlier EO plant operation and the ΔS and ΔT data obtained from the real-time data, the model, and the historical data generated during operation of the EO production system to determine how much the catalyst is overmoderated or undermoderated. The recommendation is based on the results of the overall analysis of the ΔS and ΔT data. The data processing system may determine an actionable recommendation and display the actionable recommendation on the display. For example, the data processing system may recommend that the flow rate (e.g., feed rate) of the moderator be increased relative to a current flow rate if the catalyst is undermoderated or decreased relative to the current flow rate if the catalyst is overmoderated to move the moderator level in a specific direction to reach optimum. In one embodiment, the data processing system may recommend that the moderator level (M) be increased or decreased relative to the current moderator level by a specific amount (e.g., +10%, +20%, −10%, −20%, etc.). In embodiments in which the moderator level is at optimum, the data processing system may provide a recommendation to maintain the current moderator flow rate and/or relative moderator level.

The operator of the EO production system may manually adjust the moderator level according to the recommendation provided to achieve maximum catalyst selectivity. In certain embodiments, the moderator level may be adjusted automatically. For example, a control system (e.g., the control system 108 in FIG. 13) may output a signal to a metering device (e.g., a flow control valve) that adjusts the amount of moderator 126 entering the reactor system (e.g., the reactor system 102). As discussed above, the method 200 includes providing a new moderator level setpoint to the control system. In this embodiment, the control system (e.g., the control system 108) adjusts a flow rate (e.g., feed rate) of the moderator such that, depending on the moderation level of the catalyst that is determined by the disclosed method and analysis, the amount of moderator in the feed gas is decreased or increased. The system may include an override or bypass feature that enables the operator to override/bypass the recommendation.

The method 200 disclosed herein may be iterative. As operational data for the EO production system continue to be collected and stored in the data processing system, the amount of historical data for the system increases and may be used to fine-tune the model and improve the accuracy of the estimated optimum point for maximum catalyst selectivity. The data processing system may continuously evaluate the historical data to determine the best analysis parameters that enable fine-tuning of the system to provide accurate and reliable trends. The acts of the method 200 may be repeated continuously in real-time during operation of the EO production system or on demand (e.g., initiated by the operator or when activated by a change in, for example, the EO production parameter).

As discussed above, the data processing system uses one or more decision trees to process and interpret the data (e.g., the data points 242 and 246) to assess the moderation state of the catalyst and provide an alert/recommendation and/or new moderator level setpoint.

For example, FIG. 18 illustrates the decision tree 236 that may be used by the data processing system to determine the real time value of RCl_{eff} (RCl_{eff}_{ᵣₑₐₗ₋ₜᵢₘₑ}) and appropriate alert in accordance with the present invention. In the illustrated embodiment, once the data processing determines the ΔS and ΔT for the recent historical data and the real-time data point in accordance with block 234 of FIG. 14, the decision tree 236 shown in FIG. 18 includes determining if there is a trend in the ΔS and ΔT (or RSD and RTD) data at query 284. For example, referring back to FIG. 16, a trend may be found by fitting a curve 286 to the selected data points 242.

For example, to identify a trend, the data processing system may fit the data points 242 to a polynomial or any other suitable non-linear expression having a downward concavity. Various techniques can be used to fit the curve 286 such as, but not limited to, least squares regression, non-linear regression, robust regression, weighted regression and constrained optimization, among others. If a trend is not identified, the data processing system provides an alert to proceed with an exploratory moderator step (block 287). Example 3 below provides an illustration of this decision tree result.

In certain embodiments, the data processing system may determine a confidence in the fitted curve 286 prior to proceeding to subsequent steps in the decision tree 236. That is, the data processing system may check whether the curve 286 is a good fit for the data points 242. For example, the data processing system may assess the goodness of fit of the curve 286 using a defined tunable threshold such as a minimum acceptable R 2 or adjusted R 2 metric. In one embodiment, the data processing system may check whether a real-time data point 288 matches the fitted curve 286. However, any other suitable technique may be used to assure the confidence (i.e. goodness) of the fitted curve 286 and the optimum point 294.

The fitted curve 286 includes a maximum or an optimum point 294. The optimum point 294 is defined as the point at which the difference ΔS between the measured catalyst selectivity (Sₘₑₐₛ) and the model selectivity (Sₑₛₜ) is maximized. The coordinates of this point are the optimum point (ΔTₒₚₜ, ΔSₒₚₜ) In certain embodiments, the data processing system may also determine if there is sufficient ΔS and ΔT data to determine accurately the optimum point 294. As should be appreciated, determining the maximum catalyst selectivity relative to the model estimate (ΔSₒₚₜ) (e.g., the value of ΔS at the optimum point 294) using the system and method disclosed herein does not depend on knowing the precise or accurate moderator concentration, as shown by the absence of the moderator concentration in the plot 238 illustrated in FIG. 16. That is, unlike certain existing techniques, the techniques disclosed herein do not depend on a reliable moderator concentration analysis to determine the optimum moderator level (Mₒₚₜ) for maximum catalyst selectivity (Sₒₚₜ). As illustrated in FIG. 16, the selected data points 242 encompass both sides 290, 292 of the optimum point 294, indicating that the data (e.g., the data 218, 220) in the selected period (e.g., the section 230) represent both undermoderated and overmoderated states.

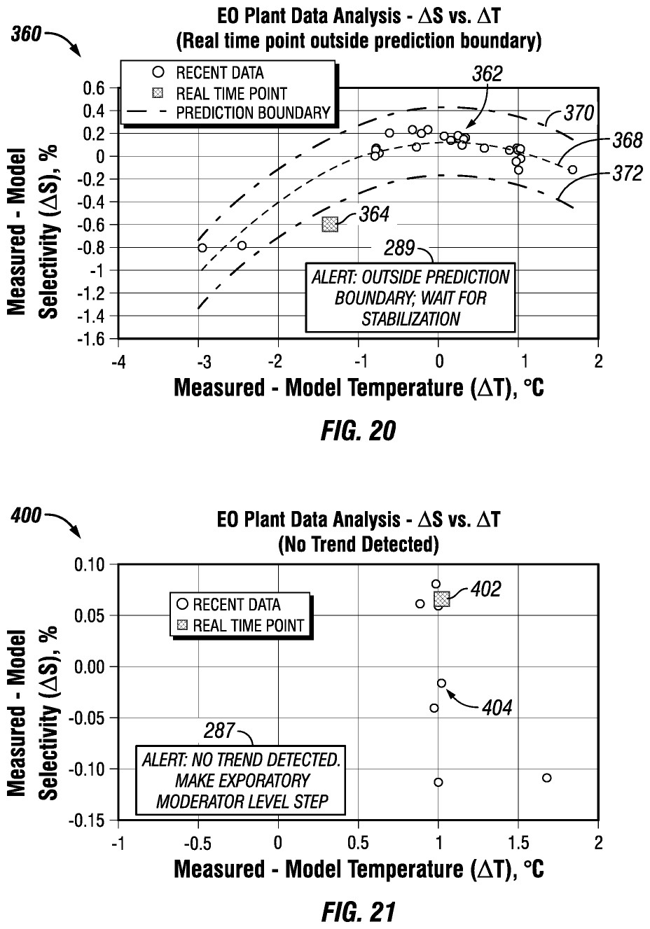

Returning to FIG. 18, following identification of the trend in accordance with query 284, the decision tree 236 includes determining if the current, real-time data point (e.g., the real-time data point 288) is within a prediction boundary at query 298. For example, the real-time data point is compared to the fitted curve to determine if it is within the prediction boundary. The prediction boundary may be defined as a fixed range that is vertically away (i.e., parallel to the y-axis) from the fitted curve known to represent typical spread in the data, or by statistical means using a standard deviation of the measurements or standard error of the fit. The prediction boundary may be within a range of approximately ±0.1 selectivity % to approximately ±0.5 selectivity %, for example, in the range of from −0.5 to +0.5 or in the range of from −0.1% to +0.1% vertically away from the fitted curve. For example, as illustrated in FIG. 16, the real-time data point 288 is essentially along the fitted curve 286 and is, therefore, within the prediction boundary. A situation where the real time data point is outside of the prediction boundary is illustrated subsequently in Example 2 (see FIG. 20).

In the decision tree 236 of FIG. 18, if the data processing system determines that the real-time data point is outside the prediction boundary, the data processing system provides an alert indicating that the real-time data point is outside the prediction boundary and to wait for stabilization of the data (block 289). However, once the data processing system determines that the real-time data point is within the prediction boundary, the data processing system proceeds to determine a slope of the fitted curve. Accordingly, the decision tree 236 includes determining a slope of the fitted curve (e.g., the slope of the ΔS vs. ΔT curve 286) for the real-time data point (block 300). For example, using the fitted curve, the slope

$$\frac{d ( Δ S )}{d ( Δ T )}$$

of the fitted curve may be determined by graphical analysis near the real-time data point, or by taking the derivative of the fitted equation and evaluating at the real-time value of ΔTᵣₑₐₗ₋ₜᵢₘₑ Referring to FIG. 16, the data points 242 along the negative slope of the curve 286 (i.e., to the right of the optimum point 294) indicate that the catalyst selectivity is not at optimum and is undermoderated. That is, the moderator level for these points is lower than the optimal moderator level. Conversely, the data points 242 along the positive slope of the curve 286 (i.e., to the left of the optimum point 294) indicate that the catalyst selectivity is not at optimum and is overmoderated. That is, the moderator level is higher than the optimal moderator level for these points. In the embodiment illustrated in FIG. 16, a slope 302 of the fitted curve 286 at the real-time data point 288 is approximately +0.28%/° C., and it is to the left of the optimum point 294, meaning it is representative of an overmoderated catalyst.

Following determination of the slope, the decision tree 236 of FIG. 18 includes determining if a real time value of the slope is within normal reference bounds at query 304. For example, the data processing system compares the real time slope value to the normal reference bounds shown in FIG. 11. By way of non-limiting example, the normal reference bounds are in the range of between approximately ±1%/° C. and approximately ±3%/° C. As defined in the plot 82 of FIG. 11, the normal reference bounds for the slope value are ±2%/° C. In the embodiment illustrated in FIG. 16, the slope 302 of the fitted curve 286 at the current real-time data point 288 is approximately +0.28%/° C., which is within the normal reference bounds of ±2%/° C. If the data processing system determines that the slope value is not within the normal reference bounds, the data processing system provides an alert that the catalyst is in an overmoderated state and to reduce the moderator level manually (block 306).

However, if the data processing system determines that the slope is within the normal reference bounds (as shown in FIG. 16), the data processing system proceeds to determine the value of RCl_{eff} for the real time point (RCl_{eff}_{ᵣₑₐₗ₋ₜᵢₘₑ}) from the slope at the real-time point and the reference curves (block 310). For example, one method to determine RCl_{eff}_{ᵣₑₐₗ₋ₜᵢₘₑ} includes comparing a local slope at the real-time data point to a reference curve. To facilitate discussion of this method, reference will be made to the FIGS. 12 and 16. As shown in FIG. 16, the value of the slope 302 at the real-time data point 288 is +0.28%/° C. This value is plotted on the curve 76 illustrated in FIG. 12 at the vertical coordinate corresponding to an estimated real-time slope value 316 of +0.28%/° C. From the plot 90 in FIG. 12 it is determined that the estimated real-time slope value 316 corresponds to a RCl_{eff} 26 of +0.080, or 8.0% overmoderated.

A second method of for determining the real-time RCl_{eff} (RCl_{eff}_{ᵣₑₐₗ₋ₜᵢₘₑ}) includes comparing the relative selectivity difference (RSD) or the relative temperature difference (RTD) with the reference curves 60, 64 (FIG. 9). In this particular method, since an optimum point is found in the fitted curve 286 of FIG. 16, it is also possible to analyze the data further to compute the RSD and the RTD using EQ. 10 and EQ. 11. This computation assures that the values of RSD and RTD are zero at the optimum point (e.g., the maximum 294), as shown in FIG. 17, and allows for easier comparison to the reference curves in FIG. 9. For example, the points in the data set 242 from the plot 238 of FIG. 16 are shown in centered format on plot 240 of FIG. 17 as the data set 246 by subtracting the values of ΔT op t and ΔSₒₚₜ at the maximum 294 (e.g., at +2.49° C., +0.53%) from the values of ΔT and ΔS, respectively, to generate the plot 240 of RSD vs. RTD illustrated in FIG. 17. The shape and slope of the plots 238, 240 are the same. However, the maximum 294 occurs at the coordinates of (0° C., 0%) rather than at the coordinates of (+2.49° C., +0.53%). In addition to removing operating condition effects, the centering of the maximum 294 at (0,0) also removes remaining impacts of the overall plant measurement biases for selectivity and temperature. This analysis leaves only the impacts of the moderator level, allowing for direct comparison of points in the data set 246 to the reference curves illustrated in FIG. 9 and facilitating determination the relative effective moderator level.

As shown in the embodiment illustrated in FIG. 17, centering the maximum 294 at the coordinates (0,0) shifts the RTD and RSD coordinates of the real-time data point 288 from (0.01° C., 0.13%) (see FIG. 16) to (−2.47° C., −0.4%). These coordinate values are compared to the respective reference curves 60, 64 illustrated in FIG. 9. For example, the comparison is first made using the RTD value (e.g., −2.47° C.), which determines the side of the curve 60 that is used to estimate the moderation level. In FIG. 9, the RTD value corresponds to a point 318 (−2.47° C.) on the reference curve 64. The point 318 lies on the right side (e.g., RCl_{eff}>0) of the maximum 72, indicating overmoderation. The corresponding RCl_{eff} at the point 318 is estimated to be +0.117, or 11.7% overmoderated.

Using knowledge that the real-time data point (e.g., the real-time data point 288) is overmoderated, the RSD value of −0.4% (the real-time data point 288 in FIG. 17) is plotted on the plot 70 on the right portion (i.e., portion that is right of the maximum 72) of the reference curve 60 at the point 320. The point 320 corresponds to an RCl_{eff} of 0.112, or 11.2% overmoderated. If the RTD value had been determined to be on the left side of maximum 72 (undermoderated), then the left side of the reference curve 60 would have been chosen to plot the RSD value.

Accordingly, for the example illustrated in FIGS. 16 and 17, three different estimates of the real-time RCl_{eff} (RCl_{eff}_{ᵣₑₐₗ₋ₜᵢₘₑ}) are obtained: (1) using the value of the ΔS vs. ΔT slope and comparison to reference curve 76 of FIG. 12, which gives RCl_{eff}=0.080, (2) using the value of RTD and comparing to reference curve 64 of FIG. 9, which gives RCl_{eff}=0.117, and (3) using the value of RSD and comparing reference curve 60 of FIG. 9, which gives RCl_{eff}=0.112. All these methods provide the same direction of moderation, with a range of between approximately 8.0% and approximately 11.7% overmoderation estimated. Various techniques can be used to select the specific value of RCl_{eff} to provide as guidance. In this embodiment, an average of the three real-time RCl_{eff} (RCl_{eff}_{ᵣₑₐₗ₋ₜᵢₘₑ}) values (e.g., 0.080, 0.117 and 0.112), which is equal to 0.103, is used for providing advice related to the moderation state of the catalyst. In certain embodiments, only one of these three different methods of determining the real-time RCl_{eff} (RCl_{eff}_{ᵣₑₐₗ₋ₜᵢₘₑ}) may be used. In other embodiments, two of these three different methods of determining the real-time RCl_{eff} (RCl_{eff}_{ᵣₑₐₗ₋ₜᵢₘₑ}) may be used. In certain embodiments, an operator of the EO production system may select which method of estimating the real-time RCl_{eff} (RCl_{eff}_{ᵣₑₐₗ₋ₜᵢₘₑ}) to use.

Following analyses and estimation of the real-time RCl_{eff} (RCl_{eff}_{ᵣₑₐₗ₋ₜᵢₘₑ}) in accordance with the acts of block 310 in FIG. 18 in one embodiment, the decision tree 236 includes using the value of the real-time RCl_{eff} (RCl_{eff}_{ᵣₑₐₗ₋ₜᵢₘₑ}) to provide a new target moderator level to the control system (block 314). The percentage change required to move the moderator level to the optimum target level is calculated using EQ. 5. With reference to the example illustrated in FIGS. 16 and 17, for an average estimated real-time RCl_{eff} (RCl_{eff}_{ᵣₑₐₗ₋ₜᵢₘₑ}) value of +0.103 obtained by averaging the three real-time RCl_{eff} (RCl_{eff}_{ᵣₑₐₗ₋ₜᵢₘₑ}) values, a change in the moderator level (M_{change}) of (1/(0.103+1)−1)*100% (a change of −9.3%, or a reduction of 9.3%) is estimated to return the moderator level to optimum. This suggested adjustment may be provided to the control system to move the moderator level to its optimum level. As should be noted, the step 314 is optional and may be omitted without departing from the scope of the present invention.

The decision tree 236 also includes evaluating the value of real-time RCl_{eff} (RCl_{eff}_{ᵣₑₐₗ₋ₜᵢₘₑ}) to determine if it is near zero at query 316. This comparison may be made using process knowledge about the precision with which the moderator level is determined and controlled. In some plants, operations may call for wider precision limits, whereas in other plants having greater control, narrower limits on precision may be employed. A near zero (i.e. near optimal) relative effective moderator level is ±0.005 to ±0.06, more specifically ±0.01 to ±0.05, and most specifically ±0.02 to ±0.04. For example, a near zero (i.e. near optimal) relative effective moderator level may be considered to be in the range of from −0.06 to +0.06, in the range of from −0.05 to +0.05, in the range of from −0.04 to +0.04, in the range of from −0.02 to +0.02, for example in the range of −0.01 to +0.01, or from −0.005 to +0.005. If the value of real-time RCl_{eff} (RCl_{eff}_{ᵣₑₐₗ₋ₜᵢₘₑ}) is within the range for a near zero RCl_{eff}, the data processing system provides an alert that the moderator level is near optimum and to hold the moderator level (block 318). However, if the value of the real-time RCl_{eff} (RCl_{eff}_{ᵣₑₐₗ₋ₜᵢₘₑ}) falls outside the range, a “NO” assessment for query 316 is determined. As such, the data processing system proceeds to determine if the estimated value of the real-time RCl_{eff} (RCl_{eff}_{ᵣₑₐₗ₋ₜᵢₘₑ}) is positive or negative at query 320.

For example, with reference to the embodiment illustrated in FIGS. 16 and 17, the average value of the real-time RCl_{eff} (RCl_{eff}_{ᵣₑₐₗ₋ₜᵢₘₑ}) is positive (+0.103), indicating an overmoderated state. The average value of the real-time RCl_{eff} (RCl_{eff}_{ᵣₑₐₗ₋ₜᵢₘₑ}) is taken by averaging the three real-time RCl_{eff} (RCl_{eff}_{ᵣₑₐₗ₋ₜᵢₘₑ}) values 0.080, 0.117 and 0.112 determined in accordance with the acts of block 310. Returning to FIG. 18, the data processing system provides an alert that the catalyst is overmoderated and recommends the moderator level be decreased by the specified amount or to a specified target (block 324). For example, the data processing system may recommend decreasing the moderator level by 9.3% in alert 324 (see FIG. 17). Equivalently, rather than specifying a percentage change in the moderator level to reach the optimum level, an absolute moderator level target for the optimum level (Mₒₚₜ) can be specified corresponding to said percentage change in moderator level. In the current example, if the catalyst chloriding effectiveness (Cl_{eff}) is chosen as the moderator level used to optimize the catalyst and its real-time value is 6.57 (Mᵣₑₐₗ₋ₜᵢₘₑ=6.57), EQ. 4 can be applied with the estimated real-time RCl_{eff} of 0.103 to compute the target optimum level:

Mₒₚₜ=Mᵣₑₐₗ₋ₜᵢₘₑ/(1+(RCl_{eff}_{ᵣₑₐₗ₋ₜᵢₘₑ})=6.57/(1+0.103)=5.96.
This change corresponds to a decrease of 9.3% in the moderator level.
Likewise, if in the current example, the make-up moderator feed rate is used as the moderator level to optimize the catalyst and its real-time value is 2.56 kg/h, EQ. 4 can be applied with the real-time RCl_{eff} of 0.103 to compute the target optimum level:

Mₒₚₜ=Mᵣₑₐₗ₋ₜᵢₘₑ/(1+RCl_{eff}_{ᵣₑₐₗ₋ₜᵢₘₑ})=2.56 kg/h/(1+0.103)=2.32 kg/h.
This change also corresponds to a decrease of 9.3% in the moderator level. This method of specifying the optimum moderator level target via the make-up moderator feed rate could be used if the gas phase chloride concentrations are not easily or accurately measurable, as can sometimes be the case in practical plant operation.
In cases where the value of the real-time RCl_{eff} is negative, indicating an undermoderated state, the data processing system provides an alert that the catalyst is undermoderated, and recommends the moderator level be increased to a specified target or by a specified amount (block 328). The alerts and/or recommendations output by the data processing system may be displayed along with the amount that that moderator level should be decreased/increased on a display (e.g., the display 192) of the EO production system (e.g., the system 100).

It should be noted that the method disclosed herein does not require precise or accurate measurement or knowledge of the gas phase moderator concentration, but rather determines direction and magnitude of the change required in the moderator level using the performance trends and model analysis. As discussed above, techniques that rely on moderator concentration measurements in the feed gas may be unreliable due to difficulties in accurate measurement and because they do not necessarily reflect the moderator concentration on the surface of the catalyst. The present invention overcomes these constraints by assessing the impact of the moderator level on the catalyst performance changes. Additionally, plotting ΔS as a function of ΔT or RSD as a function of RTD enables viewing the trends in a single trend versus two trends as shown in FIG. 15, thereby improving the user friendliness of the system.

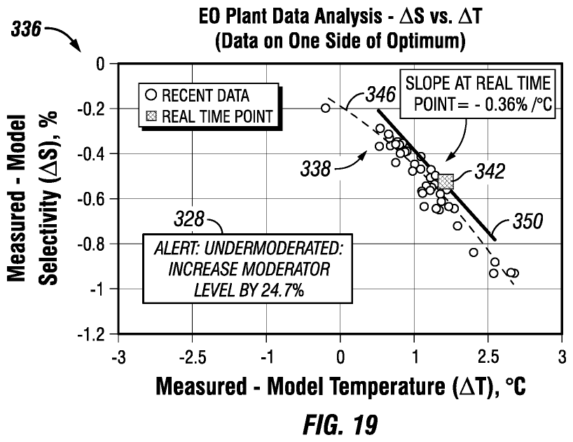

Provided below are additional examples that illustrate other potential scenarios and alerts in accordance with the decision tree 236 of FIG. 18. To facilitate discussion of the examples below reference is made to FIG. 19-23.

### EXAMPLES

### Example 1—Real-Time Point is Undermoderated, Data on One Side of Optimum

FIG. 19 illustrates a plot 336 of the ΔS vs. ΔT for a data set 338 at a selected time range. The data set 338 includes a real-time data point 342. In the illustrated example, an acceptable fitted curve is generated, and a trend 346 is found. The real-time data point 342 is assessed to determine if it is within the prediction boundary. As illustrated in FIG. 19, the real-time data point 342 is close to the fitted curve 346 and is within the prediction boundary (e.g., between approximately ±0.1% to approximately ±0.5%, for example said prediction boundary may be in the range of from −0.5 to +0.5 or in the range of from −0.1% to +0.1%). Accordingly, the data processing system determines the slope of the fitted curve 346. For example, using the fitted curve 346, a slope 350 of the curve 346 at the real-time data point 342 is determined as −0.36%/° C. This slope value is compared to the normal reference bounds (see FIG. 11), which are ±2%/° C. The calculated slope value of the curve 346 is within this range. As such, the data processing system proceeds to determine the real-time RCl_{eff}.

In the illustrated example, the data set 338 in the selected time range only lies along a region of negative slope of the fitted curve 346, which indicates the data are only on the undermoderated side of the optimum. Additionally, the fitted curve 346 does not have a maximum in the fitted data range. Therefore, only the value of the slope 350 is used to estimate RCl_{eff} using the reference curve 76 in FIG. 12. For example, referring to FIG. 12, the calculated slope of −0.36%/° C. corresponds to point 354 on the curve 76. Using the curve 76, the estimated real-time RCl_{eff} is determined to be −0.198, or 19.8% undermoderated. The percentage change required to move the moderator level to the optimum target level is calculated using EQ. 5. Therefore, the moderator level is increased by (1/(1−0.198)−1)*100% or 24.7% to return the system to optimal moderator level (e.g., block 314 in FIG. 18).

The value of the real-time RCl_{eff} is also assessed to determine if it is close to zero. For example, using a typical range of approximately ±0.02 to approximately ±0.04, as discussed above based on knowledge of the measurement and control capabilities for moderator level, the estimated RCl_{eff} of the real-time point 342 is not close to zero. Therefore, the estimated RCl_{eff} value of the real-time point 342 is further assessed to determine if the estimated real-time RCl_{eff} is positive. In this particular example, the value of the real-time RCl_{eff} is negative (−0.198). As such, the data processing system outputs the alert 328 for undermoderation and recommends increasing the moderator level by 24.7%.

### Example 2—Real Time Point is Outside the Prediction Boundary

FIG. 20 illustrates a plot 360 of the ΔS and ΔT for a data set 362 having a real-time data point 364 for a selected time range. In the illustrated example, an acceptable fit of the data set 362 is generated, and a trend 368 (i.e., fitted curve) is found.

The real-time data point 364 is assessed to determine if it falls within a prediction boundary defined by the upper and lower curves 370 and 372, respectively. As discussed above, the prediction boundary width is between approximately ±0.1% to approximately ±0.5%, for example, said boundary width may be in the range of from −0.5 to +0.5 or in the range of from −0.1% to +0.1%, relative to the fitted curve 368 and is based upon knowledge of typical variability in the data. In this particular example, the current real-time data point 364 is not between the prediction boundary curves 370 and 372, which indicates that the current real-time data point 364 is not following the same trend 368 as the other data in the data set 362 that is between the prediction boundary curves 370 and 372. This may indicate that the system is not yet stabilized. Accordingly, the data processing system outputs the alert 289 indicating that the real-time data point 364 is outside the prediction boundary, and the operator should wait for stabilization until a clearer trend is obtained.

### Example 3—No Trend Detected

FIG. 21 illustrates a plot 400 of the ΔS and ΔT for a data set 404 having a real-time data point 402. As shown in the plot 400, no acceptable trend is found for the selected data window that meets goodness of fit or range criteria. Therefore, in this particular example, the data processing system outputs the alert 287 to proceed with an exploratory moderator step.

The size and direction of such an exploratory step may be determined by the operator or control system (e.g., the control system 108) based on the EO plant's control and measurement capabilities (e.g., a change of approximately 3-5% is typical). The exploratory step allows for extending the data available and increases the likelihood of subsequently obtaining a trend that allows for better assessment of the catalyst moderation.

### Example 4—Severe Overmoderation

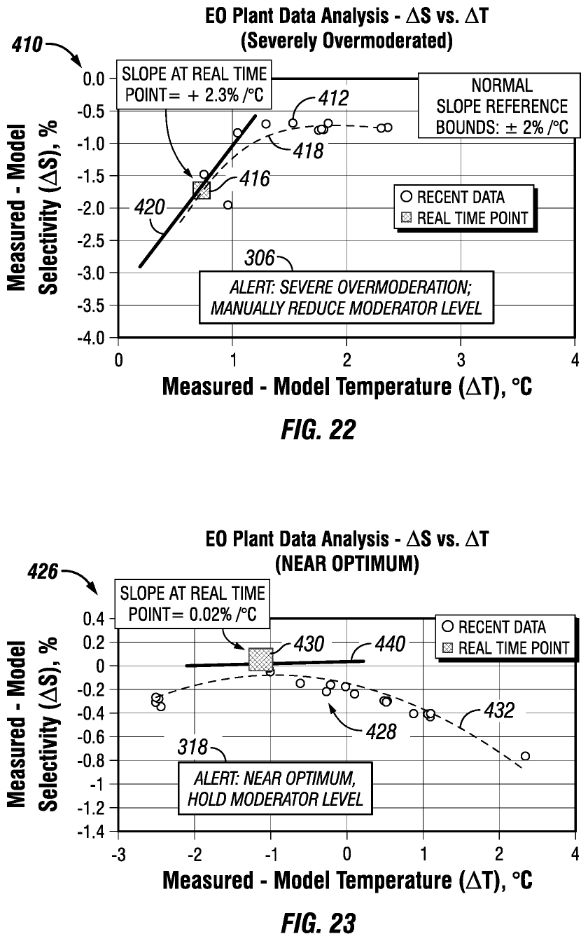

FIG. 22 illustrates a plot 410 of the ΔS and ΔT for a data set 412 having a real-time data point 416. In the illustrated example, an acceptable fit is generated and a fitted curve 418 is determined. The real-time data point 416 is near the fitted curve (e.g., within a specified range above/below the fitted curve 418, e.g., ±0.3%) and within the prediction boundary. Therefore, a local slope 420 of the fitted curve 418 is determined and evaluated at the ΔT of the current real-time data point 416 using graphical analysis or by taking the derivative of the fitted curve 418 as discussed above. The value of the slope 420 determined is +2.3%/° C. This value is compared to the normal reference bounds given in FIG. 11, which are +/−2%/° C. Comparison of the value of the slope 420 to the normal reference bounds indicates that the value is outside of this range. Accordingly, the plant operation is outside the normal range of interest, and the data processing system outputs the alert 306 that the catalyst is likely severely overmoderated (e.g., >30% overmoderated) and provides a recommendation to reduce the moderator level manually. The operator may adjust the moderator level as recommended and wait for the system to stabilize and return to the normal range where trends are clearer, and a specific moderation level target can subsequently be determined.

### Example 5—Real Time Data Point Near Optimum

FIG. 23 illustrates a plot 426 with the ΔS and ΔT for a data set 428 having a real-time data point 430. In the illustrated example, an acceptable fit is generated, and a trend 432 is found. The real-time data point 430 is close to the trend 432 (i.e., a fitted curve) and is within the prediction boundary (e.g., within approximately ±0.1% to approximately ±0.5%, for example, in the range of from −0.5 to +0.5, or in the range of from −0.1% to +0.1%). Using the fitted curve 432, a slope 440 of the curve 432 at the real-time data point 430 is determined as +0.02%/° C. The value of the slope 440 is compared to the normal reference bounds indicated in FIG. 11 (e.g., −2%/° C. to +2%/° C.). As shown, the calculated slope 440 of the curve 432 is within the normal bounds.

Therefore, a value of RCl_{eff} for the real-time point is estimated using the slope 440 and compared to the reference curve 76 in FIG. 12. The value of the estimated slope 440 is +0.02%/C and corresponds to point 442 on the reference curve 76 in FIG. 12. Using the reference curve 76, the estimated RCl_{eff} for the real-time point is determined to be +0.011, or 1.1% overmoderated. This is within the normal range of ±0.02 to ±0.04, which is considered to be within the measurement and control noise of zero for the RCl_{eff}. Therefore, the data processing system outputs the alert 318 indicating that the catalyst is near optimum and recommends maintaining the moderator level.

As discussed above, the techniques disclosed herein may be used to determine optimum moderator levels to achieve maximum catalyst selectivity in a reliable and robust manner. The system and method use a combination of reference data, historical data and model data to determine optimum moderator levels and catalyst performance without relying on monitoring precise and accurate moderator concentrations. The disclosed system and method may provide alerts/recommendations in real-time on how to adjust moderator levels to reach maximum catalyst selectivity. By using historical data specific to the EO production system and catalyst specific data, the model may be fine-tuned and provide accurate and reliable estimates of the optimum moderator levels and maximum catalyst selectivity. In this way, disadvantages associated with existing techniques that rely on stepping moderator levels to determine the optimum and monitoring gas phase moderator concentrations, which might not be indicative of the amount of moderator on the surface of the catalyst, may be mitigated. Accordingly, by using the disclosed system and method, the accuracy and reliability of the optimum moderator levels that achieve maximum catalyst selectivity may be improved compared to existing techniques that rely on the concentration of the moderator to determine maximum catalyst selectivity. Additionally, the disclosed system and method provide actionable guidance/recommendations about both the direction and magnitude of adjustments required to achieve maximum catalyst selectivity, or if more data are needed to gain a usable trend.

The present invention may be embodied in other specific forms without departing from its spirit or essential characteristics. The described embodiments are to be considered in all respects only as illustrative and not restrictive.

1. A method for maximizing the selectivity (S) of an epoxidation catalyst in an ethylene oxide reactor system, comprising:
receiving a measured reactor selectivity (Sₘₑₐₛ), a measured reactor temperature (Tₘₑₐₛ), and one or more operational parameters from an ethylene oxide production system configured to convert, in the ethylene oxide reactor system, a feed gas comprising ethylene and oxygen into ethylene oxide in the presence of the epoxidation catalyst and a chloride-containing catalyst moderator, wherein the epoxidation catalyst comprises silver and a promoting amount of rhenium (Re), and wherein the measured reactor selectivity (Sₘₑₐₛ), the measured reactor temperature (Tₘₑₐₛ), and the one or more operational parameters comprise real-time and historical operating data points over time generated by the ethylene oxide production system; and
using a processor to:
(a) calculate, using a model, for each time point, a model-estimated selectivity (Sₑₛₜ) and a model-estimated temperature (Tₑₛₜ) of the epoxidation catalyst at an optimum moderator level (Mₒₚₜ), wherein the model-estimated selectivity (Sₑₛₜ) and the model-estimated temperature (Tₑₛₜ) are determined based on at least one operational parameter of the one or more operational parameters at said time points, wherein the at least one operational parameter does not include a chloride-containing moderator level, and wherein the model is based, at least in part, on empirical historical data associated with the epoxidation catalyst, the ethylene oxide production system, or both;
(b) determine a difference (ΔS) between the measured reactor selectivity (Sₘₑₐₛ) and the model-estimated selectivity (Sₑₛₜ) and a difference (ΔT) between the measured reactor temperature (Tₘₑₐₛ) and the model-estimated temperature (Tₑₛₜ) for each of the time points;
(c) fit a curve to the delta selectivity (ΔS) data points as a function of the corresponding delta temperature (ΔT) data points to obtain a fitted curve;
(d) determine a real-time relative effective moderator level (RCl_{eff}_{ᵣₑₐₗ₋ₜᵢₘₑ}) based on the fitted curve and real-time values of ΔS (ΔSᵣₑₐₗ₋ₜᵢₘₑ) and ΔT (ΔTᵣₑₐₗ₋ₜᵢₘₑ);
(e) output an actionable recommendation based on the real-time RCl_{eff} (RCl_{eff}_{ᵣₑₐₗ₋ₜᵢₘₑ}), wherein the recommendation comprises a target change (M_{change}) of a moderator level (M) to its optimal value (Mₒₚₜ) such that the RCl_{eff} is changed from its real-time value (RCl_{eff}_{ᵣₑₐₗ₋ₜᵢₘₑ}) to the optimum level of 0.0 by definition or the equivalent absolute moderator level target (Mₒₚₜ); and
(f) display the actionable recommendation on a display;
wherein
the RCl_{eff} is defined to be the value of the ratio of the moderator level (M) to the optimum moderator level (Mₒₚₜ) minus one:
RCl_{eff}=(M/Mₒₚₜ)−1
and, wherein the moderator level (M) is defined as a total or weighted total concentration of chloride species in the feed gas to the ethylene oxide reactor system, a makeup feed rate of chlorides or a catalyst chloriding effectiveness value (Cl_{eff}), which is calculated as:
$${Cl}_{eff} = \frac{( 0.1 * [ MC ] + [ EC ] + 2 * [ EDC ] + [ VC ] )}{( 0.002 * [ {CH}_{4} ] + [ {C}_{2} {H}_{6} ] + 0.01 * [ {C}_{2} {H}_{4} ] )}$$
whereby [MC], [EC], [EDC], and [VC] are the concentrations in ppmv of methyl chloride (MC), ethyl chloride (EC), ethylene dichloride (EDC), and vinyl chloride (VC), respectively, and [CH₄], [C₂H₆] and [C₂H₄] are the concentrations in mole percent of methane, ethane, and ethylene, respectively, in the feed gas,
wherein the recommended change to bring the moderator level (M) from its real-time level (Mᵣₑₐₗ₋ₜᵢₘₑ) to its optimal level (Mₒₚₜ) and to bring the RCl_{eff} to its optimum level of 0.0 is defined as
M_{change}=(1/(RCl_{eff}_{ᵣₑₐₗ₋ₜᵢₘₑ}+1)−1)*100%,
in percentage terms or, as the equivalent incremental change in moderator level, or equivalently, wherein the absolute recommended optimal moderator level target (Mₒₚₜ) is defined as
Mₒₚₜ=Mᵣₑₐₗ₋ₜᵢₘₑ/(RCl_{eff}_{ᵣₑₐₗ₋ₜᵢₘₑ}+1)
and
wherein the real-time RCl_{eff} (RCl_{eff}_{ᵣₑₐₗ₋ₜᵢₘₑ}) is determined by:
(i) determining a slope of the fitted curve at the real-time values of ΔS (ΔSᵣₑₐₗ₋ₜᵢₘₑ) and ΔT (ΔTᵣₑₐₗ₋ₜᵢₘₑ) and comparing the slope to a reference curve for the epoxidation catalyst, or by
(ii) determining from the fitted curve a maximum ΔS (ΔSₒₚₜ) and a corresponding ΔT (ΔTₒₚₜ) at the maximum ΔS, wherein the ΔSₒₚₜ occurs at the optimum RCl_{eff}, calculating a relative selectivity difference (RSD) by subtracting the ΔSₒₚₜ from the ΔS and a relative temperature difference (RTD) by subtracting the ΔTₒₚₜ from the ΔT, and comparing real-time values of the RSD (RSDᵣₑₐₗ₋ₜᵢₘₑ) and the RTD (RTDᵣₑₐₗ₋ₜᵢₘₑ) to reference curves for the epoxidation catalyst, or by a combination of said methods (i) and (ii),
wherein the reference curves are generated from previous laboratory testing, pilot plant testing, or earlier plant operation that relate the selectivity deviations and temperature deviations versus optimum to the relative effective moderator level (RCl_{eff}) or that relate the slope of the plot of said selectivity deviations plotted against said temperature deviations to the relative effective moderator level (RCl_{eff}).

2. The method of claim 1, wherein the slope of the fitted curve at the real-time values of ΔS (ΔSᵣₑₐₗ₋ₜᵢₘₑ) and ΔT (ΔTᵣₑₐₗ₋ₜᵢₘₑ) is within normal reference bounds, and wherein the normal reference bounds are within a range of ±1%/° C. to ±3%/° C.

3. The method of claim 1, wherein the real-time RCl_{eff} (RCl_{eff}_{ᵣₑₐₗ₋ₜᵢₘₑ}) is at the optimum RCl_{eff} when it is at or near zero, wherein at or near zero is within the range ±0.01 to ±0.05, wherein the epoxidation catalyst is overmoderated when RCl_{eff}_{ᵣₑₐₗ₋ₜᵢₘₑ} is positive and not at or near zero, and wherein the epoxidation catalyst is undermoderated when RCl_{eff}_{ᵣₑₐₗ₋ₜᵢₘₑ} is negative and not at or near zero.

4. The method of claim 1, wherein the real-time value of ΔS (ΔSᵣₑₐₗ₋ₜᵢₘₑ) is within a prediction boundary of the fitted curve, and wherein the prediction boundary is within the range of ±0.1% to ±0.5%.

5. The method of claim 1, comprising making a target change (M_{change}) of a moderator level (M) to its optimal value (Mₒₚₜ) such that the RCl_{eff} is changed from its real-time value (RCl_{eff}_{ᵣₑₐₗ₋ₜᵢₘₑ}) to the optimum level of 0.0.

6. The method of claim 1, comprising triggering an alarm when the real-time RCl_{eff} (RCl_{eff}_{ᵣₑₐₗ₋ₜᵢₘₑ}) in the ethylene oxide reactor system is not at the optimum Rcl_{eff} or not within a range of ±0.01 to ±0.05 of the optimum Rcl_{eff}.

7. The method of claim 1, wherein the one or more operational parameters comprises gas hourly space velocity (GHSV), pressure, the moderator level, feed gas composition, EO production parameter and combinations thereof, wherein the EO production parameter is selected from the group comprising product gas ethylene oxide concentration, a change in the number of moles of EO produced from an inlet to an outlet of a reactor in the ethylene oxide reactor system, an ethylene oxide production rate, an ethylene oxide production rate per mass of silver loaded into the reactor, an ethylene oxide production rate per catalyst mass and the work rate.

8. One or more tangible, non-transitory, machine-readable media configured to maximize a selectivity (S) of an epoxidation catalyst in an ethylene oxide reactor system and comprising instructions to:
(a) calculate, using a model, for real-time and historical points over time, a model-estimated selectivity (Sₑₛₜ) and a model-estimated temperature (Tₑₛₜ) of the epoxidation catalyst at an optimum moderator level (Mₒₚₜ) based on at least one operational parameter at said time points from an ethylene oxide production system comprising the ethylene oxide reactor system, wherein the model is based, at least in part, on empirical historical data associated with the epoxidation catalyst, the ethylene oxide production system, or both, wherein the at least one operational parameter does not include a chloride-containing moderator level, and wherein the epoxidation catalyst comprises silver and a promoting amount of rhenium (Re);
(b) determine a difference (ΔS) between a measured reactor selectivity (Sₘₑₐₛ) and the model-estimated selectivity (Sₑₛₜ) and a difference (ΔT) between a measured reactor temperature (Tₘₑₐₛ) and the model-estimated temperature (Tₑₛₜ) for each of the time points, wherein the measured reactor selectivity (Sₘₑₐₛ) the measured reactor temperature (Tₘₑₐₛ) comprise real-time and historical operating data points over time generated by the ethylene oxide production system at said time points;
(c) fit a curve to the delta selectivity (ΔS) data points as a function of the corresponding delta temperature (ΔT) data points to obtain a fitted curve;
(d) determine a real-time relative effective moderator level (RCl_{eff}_{ᵣₑₐₗ₋ₜᵢₘₑ}) based on the fitted curve and real-time values of ΔS (ΔSᵣₑₐₗ₋ₜᵢₘₑ) and ΔT (ΔTᵣₑₐₗ₋ₜᵢₘₑ);
(e) output an actionable recommendation based on the real-time RCl_{eff} (RCl_{eff}_{ᵣₑₐₗ₋ₜᵢₘₑ}), wherein the recommendation comprises a target change (M_{change}) of a moderator level (M) to its optimal value (Mₒₚₜ) such that the RCl_{eff} is changed from its real-time value (RCl_{eff}_{ᵣₑₐₗ₋ₜᵢₘₑ}) to the optimum level of 0.0 by definition or the equivalent absolute moderator target (Mₒₚₜ); and
(f) display the actionable recommendation on a display;
wherein
the RCl_{eff} is defined to be the value of the ratio of the moderator level (M) to the optimum moderator level (Mₒₚₜ) minus one:
RCl_{eff}=(M/Mₒₚₜ)−1
and, wherein the moderator level (M) is defined as a total or weighted total concentration of chloride species in the feed gas to the ethylene oxide reactor system, a makeup feed rate of chlorides, or a catalyst chloriding effectiveness value (Cl_{eff}), which is calculated as:
$${Cl}_{eff} = \frac{( 0.1 * [ MC ] + [ EC ] + 2 * [ EDC ] + [ VC ] )}{( 0.002 * [ {CH}_{4} ] + [ {C}_{2} {H}_{6} ] + 0.01 * [ {C}_{2} {H}_{4} ] )}$$
whereby [MC], [EC], [EDC], and [VC] are the concentrations in ppmv of methyl chloride (MC), ethyl chloride (EC), ethylene dichloride (EDC), and vinyl chloride (VC), respectively, and [CH₄], [C₂H₆] and [C₂H₄] are the concentrations in mole percent of methane, ethane, and ethylene, respectively, in the feed gas,
wherein the recommended change to bring the moderator level (M) from its real-time level (Mᵣₑₐₗ₋ₜᵢₘₑ) to its optimal level (Mₒₚₜ) and to bring the RCl_{eff} to its optimum level of 0.0 is defined as
M_{change}=(1/(RCl_{eff}_{ᵣₑₐₗ₋ₜᵢₘₑ}+1)−1)*100%,
in percentage terms or, as the equivalent incremental change in moderator level, or, equivalently, wherein the absolute recommended optimal moderator level target (Mₒₚₜ) is defined as
Mₒₚₜ=Mᵣₑₐₗ₋ₜᵢₘₑ/(RCl_{eff}_{ᵣₑₐₗ₋ₜᵢₘₑ}+1),
wherein the real-time RCl_{eff} (RCl_{eff}_{ᵣₑₐₗ₋ₜᵢₘₑ}) is determined by:
(i) determining a slope of the fitted curve at the real-time values of ΔS (ΔSᵣₑₐₗ₋ₜᵢₘₑ) and ΔT (ΔTᵣₑₐₗ₋ₜᵢₘₑ) and comparing the slope to a reference curve for the epoxidation catalyst, or by
(ii) determining from the fitted curve a maximum ΔS (ΔSₒₚₜ) and a corresponding ΔT (ΔTₒₚₜ) at the maximum ΔS, wherein the maximum ΔS (ΔSₒₚₜ) occurs at the optimum RCl_{eff}, calculating a real-time relative selectivity difference (RCl_{eff}_{ᵣₑₐₗ₋ₜᵢₘₑ}) by subtracting the ΔSₒₚₜ from the ΔSᵣₑₐₗ₋ₜᵢₘₑ and a real-time relative temperature difference (RTDᵣₑₐₗ₋ₜᵢₘₑ) by subtracting the ΔTₒₚₜ from the ΔTᵣₑₐₗ₋ₜᵢₘₑ, and comparing a real-time value of the RSD (RSDᵣₑₐₗ₋ₜᵢₘₑ) and the RTD (RTDᵣₑₐₗ₋ₜᵢₘₑ) to reference curves for the epoxidation catalyst,
or by a combination of said methods (i) and (ii),
wherein the reference curves are generated from previous laboratory testing, pilot plant testing, or earlier plant operation that relate the selectivity deviations and temperature deviations versus optimum to the relative effective moderator level (RCl_{eff}) or that relate the slope of the plot of said selectivity deviations plotted against said temperature deviations to the relative effective moderator level (RCl_{eff}).

9. The one or more machine-readable media of claim 8, wherein the real-time RCl_{eff} (RCl_{eff}_{ᵣₑₐₗ₋ₜᵢₘₑ}) is at the optimum RCl_{eff} when it is at or near zero, wherein at or near zero is defined as being within a range of ±0.01 to ±0.05, and wherein the epoxidation catalyst is overmoderated when the real-time RCl_{eff} (RCl_{eff}_{ᵣₑₐₗ₋ₜᵢₘₑ}) is positive and not at or near zero, and wherein the epoxidation catalyst is undermoderated when the real-time RCl_{eff} (RCl_{eff}_{ᵣₑₐₗ₋ₜᵢₘₑ}) is negative and not at or near zero.

10. The one or more machine-readable media of claim 8, comprising instructions to make a target change (M_{change}) of a moderator level (M) from its real-time level (Mᵣₑₐₗ₋ₜᵢₘₑ) to its optimal value (Mₒₚₜ) such that the RCl_{eff} is changed from its real-time value to the optimum level of 0.0, or equivalently, instructions to make a change of the moderator level to an absolute target optimal moderator level (Mₒₚₜ).

11. The one or more machine-readable media of claim 8, comprising instructions to trigger an alarm when the real-time RCl_{eff} (RCl_{eff}_{ᵣₑₐₗ₋ₜᵢₘₑ}) in the ethylene oxide reactor system is not at the optimum RCl_{eff} or within a range of ±0.01 and ±0.05 of the optimum RCl_{eff}.

12. A system, comprising:
a reactor disposed in an ethylene oxide production system and comprising ethylene, oxygen, an epoxidation catalyst, and a chloride-containing catalyst moderator, wherein the reactor is configured to convert the ethylene and the oxygen into ethylene oxide, and wherein the epoxidation catalyst comprises silver and a promoting amount of rhenium (Re);
a display; and
a data processing system configured to receive a measured reactor selectivity (Sₘₑₐₛ), a measured reactor temperature (Tₘₑₐₛ), and one or more operational parameters from the ethylene oxide production system, wherein the measured reactor selectivity (Sₘₑₐₛ), the measured reactor temperature (Tₘₑₐₛ), and the one or more operational parameters comprise real-time and historical operating data points over time generated by the ethylene oxide production system, and wherein the data processing system comprises a processor and one or more tangible, non-transitory, machine-readable media comprising instructions that when executed by the processor are configured to:
(a) calculate, using a model, for each time point, a model-estimated selectivity (Sₑₛₜ) and a model-estimated temperature (Tₑₛₜ) of the epoxidation catalyst at an optimum moderator level (Mₒₚₜ), wherein the model-estimated selectivity (Sₑₛₜ) and temperature (Tₑₛₜ) are determined based on at least one operational parameter of the one or more operational parameters at said time points, wherein the at least one operational parameter does not include a chloride-containing moderator level, and wherein the model is based at least in part on empirical historical data associated with the epoxidation catalyst, the ethylene oxide production system, or both;
(b) determine the difference (ΔS) between the measured reactor selectivity (Sₘₑₐₛ) and the model-estimated selectivity (Sₑₛₜ) and the difference (ΔT) between the measured reactor temperature (Tₘₑₐₛ) and the model-estimated temperature (Tₑₛₜ) for each of the time points;
(c) fit a curve to the delta selectivity (ΔS) data points as a function of the corresponding delta temperature (ΔT) data points to obtain a fitted curve;
(d) determine a real-time relative effective moderator level (RCl_{eff}_{ᵣₑₐₗ₋ₜᵢₘₑ}) based on the fitted curve and real-time values of ΔS (ΔS and ΔT (ΔTᵣₑₐₗ₋ₜᵢₘₑ);
(e) output an actionable recommendation based on the real-time RCl_{eff} (RCl_{eff}_{ᵣₑₐₗ₋ₜᵢₘₑ}), wherein the recommendation comprises a target change (M_{change}) of a moderator level (M) to its optimal value (Mₒₚₜ) such that the RCl_{eff} is changed from its real-time value (RCl_{eff}_{ᵣₑₐₗ₋ₜᵢₘₑ}) to the optimum level of 0.0 by definition, or the equivalent absolute moderator level target (Mₒₚₜ); and
(f) display the actionable recommendation on a display;
wherein
the RCl_{eff} is defined to be the value of the ratio of the moderator level (M) to the optimum moderator level (Mₒₚₜ) minus one:
RCl_{eff}=(M/Mₒₚₜ)−1
and, wherein the moderator level (M) is defined as a total or weighted total concentration of chloride species in the feed gas to the ethylene oxide reactor system, a makeup feed rate of chlorides or a catalyst chloriding effectiveness value (Cl_{eff}), which is calculated as:
$${Cl}_{eff} = \frac{( 0.1 * [ MC ] + [ EC ] + 2 * [ EDC ] + [ VC ] )}{( 0.002 * [ {CH}_{4} ] + [ {C}_{2} {H}_{6} ] + 0.01 * [ {C}_{2} {H}_{4} ] )}$$
whereby [MC], [EC], [EDC], and [VC] are the concentrations in ppmv of methyl chloride (MC), ethyl chloride (EC), ethylene dichloride (EDC), and vinyl chloride (VC), respectively, and [CH₄], [C₂H₆] and [C₂H₄] are the concentrations in mole percent of methane, ethane, and ethylene, respectively, in the feed gas,
wherein the recommended change to bring the moderator level (M) from its real-time level (Mᵣₑₐₗ₋ₜᵢₘₑ) to its optimal level (Mₒₚₜ) and to bring the RCl_{eff} to its optimum level of 0.0 is defined as
M_{change}=(1/(RCl_{eff}_{ᵣₑₐₗ₋ₜᵢₘₑ}+1)−1)*100%,
in percentage terms or, as the equivalent incremental change in moderator level, or equivalently, wherein the absolute recommended optimal moderator level target is defined as
Mₒₚₜ=Mᵣₑₐₗ₋ₜᵢₘₑ/(RCl_{eff}_{ᵣₑₐₗ₋ₜᵢₘₑ}+1),
wherein the real-time RCl_{eff} (RCl_{eff}_{ᵣₑₐₗ₋ₜᵢₘₑ}) is determined by:
(i) determining a slope of the fitted curve at the real-time values of ΔS (ΔSᵣₑₐₗ₋ₜᵢₘₑ) and ΔT (ΔTᵣₑₐₗ₋ₜᵢₘₑ) and comparing the slope to a reference curve for the epoxidation catalyst, or by
(ii) determining from the fitted curve a maximum ΔS (ΔSₒₚₜ) and a corresponding ΔT (ΔTₒₚₜ) at the maximum ΔS, wherein the maximum ΔS occurs at the optimum RCl_{eff}, calculating a relative selectivity difference (RSD) by subtracting the ΔSₒₚₜ from the ΔS and a relative temperature difference (RTD) by subtracting the ΔTₒₚₜ from the ΔT, and comparing real-time values of the RSD (RSDᵣₑₐₗ₋ₜᵢₘₑ) and the RTD (RTDᵣₑₐₗ₋ₜᵢₘₑ) to reference curves for the epoxidation catalyst, or by a combination of said methods (i) and (ii),
wherein the reference curves are generated from previous laboratory testing, pilot plant testing, or earlier plant operation that relate the selectivity deviations and temperature deviations versus optimum to the relative effective moderator level (RCl_{eff}) or that relate the slope of the plot of said selectivity deviations plotted against said temperature deviations to the relative effective moderator level (RCl_{eff}).

13. The system of claim 12, wherein the real-time RCl_{eff} (RCl_{eff}_{ᵣₑₐₗ₋ₜᵢₘₑ}) is at the optimum RCl_{eff} when it is at or near zero, wherein at or near zero is defined as being within a range of ±0.01 to ±0.05, wherein the epoxidation catalyst is overmoderated when the real-time RCl_{eff} (RCl_{eff}_{ᵣₑₐₗ₋ₜᵢₘₑ}) is positive and not at or near zero, and wherein the epoxidation catalyst is undermoderated when the real-time RCl_{eff} (RCl_{eff}_{ᵣₑₐₗ₋ₜᵢₘₑ}) is negative and not at or near zero.

14. The system of claim 12, wherein the data processing system is configured to trigger an alarm when the real-time RCl_{eff} (RCl_{eff}_{ᵣₑₐₗ₋ₜᵢₘₑ}) in the ethylene oxide reactor system is not at the optimum RCl_{eff} or not within a range of ±0.01 and ±0.05 of the optimum RCl_{eff}.
